(19) United States
(12) Patent Application Publication
(10) Pub. No.: US 2021/0181183 A1
(43) Pub. Date: Jun. 17, 2021

(54) METHODS FOR DETERMINING SELECTIVITY OF TEST COMPOUNDS

(71) Applicant: CEMM - FORSCHUNGSZENTRUM FÜR MOLEKULARE MEDIZIN GmBH, Wien (AT)

(72) Inventors: Nikolaus KRALL, Emmersdorf a.d. Donau (AT); Giulio SUPERTI-FURGA, Wien (AT); Gregory VLADIMER, Wien (AT)

(73) Assignee: CEMM - FORSCHUNGSZENTRUM FÜR MOLEKULARE MEDIZIN GmBH, Wien (AT)

(21) Appl. No.: 16/760,222

(22) PCT Filed: Oct. 30, 2018
(86) PCT No.: PCT/EP2018/079746

§ 371 Date: Apr. 29, 2020

(30) Foreign Application Priority Data
Oct. 31, 2017 (EP) 17199353.8

(51) Int. Cl. G01N 33/50

(52) U.S. Cl. CPC G01N 33/5011; G01N 33/5008

(57) ABSTRACT

The invention relates to methods for determining the selectivity of a test compound and related methods such as methods for determining whether a subject suffering from cancer will respond or is responsive to treatment with a test compound or compositions comprising more than one test compound.

The invention relates to methods for determining the selectivity of a test compound and related methods such as methods for determining whether a subject suffering from cancer will respond or is responsive to treatment with a test compound. In particular methods comprising the steps of (a) providing a sample comprising at least two distinguishable sub-populations of cells in a total population of cells; (b) dividing the sample into at least two parts; (c) incubating at least one part obtained in (b) in the absence of a test compound and at least one part obtained in (b) in the presence of a test compound; (d) determining the number of cells in one of the at least two sub-populations that exhibit a distinguishable phenotype, relative to the number of cells in the total population of cells that exhibit the same phenotype in (i) the at least one part incubated in the presence of the test compound and (ii) in the at least one part incubated in the absence of the test compound; and (e) determining selectivity of the test compound to induce the phenotype referred to in (d) in the one sub population referred to in step (d) over all other subpopulations by dividing (i) through (ii) wherein the test compound selectively induces the phenotype referred to in (d) if (i) divided through (ii) is greater than 1, preferably greater than 1.05, 1.1, 1.5, 2, 3 most preferably 5, and selectively inhibits or reduces the phenotype referred to in (d) if it is less than 1, preferably less than 0.95, 0.9, 0.7, 0.5, 0.3, most preferably less than 0.2. The invention also provides for methods for determining whether a subject suffering from cancer will respond or is responsive to treatment with a test compound, wherein the method comprises (a) providing a sample from the subject comprising at least two sub-populations of cells in a total population cells, wherein at least one sub-population corresponds to cancerous cells and at least one sub-populations corresponds to non-cancerous cells; (b) dividing the sample into at least two parts; (c) incubating at least one part obtained in (b) in the absence of a test compound and at least one part in the presence of a test compound; (d) determining the number of viable cells in at least one of the sub-populations corresponding to cancer cells relative to the number of viable cells in the total population of cells in (i) the at least one part incubated in the presence of the test compound and (ii) the at least one part incubated in the absence of the test compound; and (e) determining whether the subject will respond or is responsive to treatment with the test compound by dividing (i) through (ii), wherein the subject will respond or is responsive to treatment if the resulting value is less than 1, preferably less than 0.95, 0.9, 0.8, 0.6, 0.4 most preferably less than 0.2.

The identification of drugs for the treatment of human and/or animal diseases requires the identification of molecules that selectively induce a desired biological effect in particular cell types while not affecting other cells thus causing unwanted side effects. Conversely, a drug that is capable of selectively inducing a desired biological effect in the desired cell type in an individual patient is likely to provide said patient with a medical benefit. A drug with lower selectivity may cause severe side effects, may require the reduction of the treatment dose and may consequently not lead to the desired medical outcome.

In the past, drug discovery has relied heavily on the use of cell line model systems. It is increasingly understood, however, that these models only incompletely recapitulate complex processes of higher organisms which often involve the interplay of different cell types. In particular, selectivity as the ability to induce an effect only in the desired target cell type cannot be studied in such systems. A possible solution practiced in the art is to test molecules in different cell lines independently. However, this does not take into account the possible interplay of different cell types. Alternatives are the establishment of co-culture model systems in which different cell lines are mixed to recapitulate a more realistic environment. Additionally, chemical matter can also be tested directly in primary material such as PBMCs or bone marrow comprised of multiple cell types ex vivo. In the context of drug discovery and development, efficient methods are thus required to measure how chemical matter selectively affects one cell type over others in mixtures of cells comprising at least two different cell types.

It is also becoming increasingly clear that different patients suffering from the same medical condition may have vastly different responses to the same medicament. It is estimated that up to 90% of all prescribed drugs only benefit 25% of the patients.

Treating a patient with a likely ineffective medicament may not only cause unnecessary suffering due to side effects and lack of medical benefit but will also waste precious healthcare financial resources. Methods are thus required to accurately predict treatment outcome for individual patients in order to enable physicians to give the right drug to the right patient at the right time.

Methods of the art for the personalized prediction of treatment response can broadly be divided into methods that infer treatment response from indirect biomarkers (e.g., inferring from a mutation in BCR-ABL that a patient will likely not respond to Imatinib) and methods that directly measure drug action in primary patient material to predict treatment outcome (i.e., functional ex vivo drug tests).

Functional ex vivo drug tests known in the art include the MiCK assay (Kravtsov et al., Blood. 92 (3): 968-80), the method described in US20100298255A1, or the Cell Titer Glo assay of Promega Coporation. These methods focus on testing whether a target cell population extracted from a patient is responsive to a particular medicament under suitable ex vivo incubation conditions. Here, we present evidence, that unlike prior belief in the field, it is insufficient to merely measure drug response in the target cell population but in fact the selectivity of a drug to affect target cells as opposed to off-target cells is essential to predict treatment outcome (Examples 2-4). Thus, also for the prediction of clinical drug response using functional ex vivo drug response tests, efficient means are required to measure how chemical matter (e.g. FDA approved drugs) selectively affect one cell population over others in mixtures of cells comprising at least two different sub-population of cells.

Selectivity of a drug to affect one cell population over another population is traditionally measured by comparing the concentration at which 50% of the desired or on-target effect is achieved (EC50) in the cell population of interest to the concentration at which 50% of the same effect is achieved in the off-target population of cells.

One approach practiced in the art is to isolate target and off-target cell populations or provide isolated cell lines representing target and off-target cell populations and individually measure drug response over different concentrations in these isolated cell populations. The disadvantage of this approach is that target cells are not analysed in their natural environment, which may affect the outcome in various ways. Moreover, if target cells need to be isolated from complex cell mixtures (e.g., PBMCs), this manipulation introduces additional perturbation to the system that may affect the measurement outcome. Further, effects that are dependent on cell-cell interactions through direct physical interactions or action at a distance through soluble messengers (e.g., cells of the immune system clearing damaged but not apoptotic cancer cells) cannot be reproduced in isolated cell model systems.

In order to determine selectivity of drugs or other chemical test compounds in complex mixtures comprised of two or more cell types, novel methods are required to distinguish between different cell populations and selectively measure drug effect on each cell population individually. The standard method practiced in the art to analyse complex cell mixtures is flow cytometry. Here, individual cell populations can be distinguished and the effect of a test compound can be measured using fluorescent dyes and markers (e.g., through staining with fluorescently labelled antibodies, fluorescent live-death staining). Another approach has been described in WO 2016/046346.

In order to determine the EC50 of a chemical test compound such as a drug acting on a particular cell sub-population in a complex mixture of cells using flow cytometry, the person skilled in the art would count the cells of the sub-population of interest (i.e., determine the absolute cell number) exhibiting the desired phenotype upon treatment with the chemical compound at typically 4 or more concentrations of the chemical compound, plot the number of cells exhibiting the desired phenotype upon treatment with the chemical compound against concentration of the chemical compound and fit a 4 parameter logistic or other sigmoidal model to determine the EC50. Alternatively, a proxy measure that is directly proportional to the phenotype such as ATP levels for total number of viable cells can be used. This is evidenced by numerous prior art documents and application notes such as Hernandez et al., SLAS Technology 2017, Vol. 22(3) 325-337 (here: phenotype is live leukemic cells) or Ross et al. Cancer Research 49, 3776-3782. Jul. 15, 1989 and the considerable effort that has been invested into validating and optimizing the ability to count absolute cell numbers using flow cytometry.

In order to determine selectivity of a test compound to affect one cell type over another in a complex mixture of cells, the person skilled in the art would consequently measure the EC50 of the test compound towards both cell populations, take the ratio of the the EC50 values or the difference of the log EC50 values as the measure of selectivity (c.f., definition of therapeutic index used by FDA).

However, this approach suffers from major limitations: Absolute cell numbers are inherently difficult to measure using flow cytometry and also other single cell analysis techniques. Absolute cell numbers seeded into assay plates may vary significantly in particular when automated cell dispensing machinery is used. During subsequent staining and washing steps, cells may be lost differentially from different assay wells. Further, absolute cell quantification typically needs to be benchmarked against bead standards which introduces another source of error and represents additional effort. Finally, determination of selectivity will require the measurement of test compound effect at different test compound concentrations which vastly increases the sample requirement and assay time.

In view of the foregoing, there is a need in the art for methods for determining selectivity of chemical compounds in complex cellular mixtures.

The technical problem to be solved by the present invention is thus the provision of improved methods for determining selectivity of one or more test compound(s) and related methods based on the improved determined selectivity.

The invention; in a first embodiment, thus relates to a method for determining the selectivity of a test compound, the method comprising the steps:
(a) providing a sample comprising at least two distinguishable sub-populations of cells in a total population of cells;
(b) dividing the sample into at least two parts;
(c) incubating at least one part obtained in step (b),in the absence of a test compound and at least one part obtained in step (b) in the presence of a test compound;
(d) determining the number of cells in one of the at least two sub-populations that exhibit a distinguishable phenotype, relative to the number of cells in the total population of cells that exhibit the same phenotype in
   (i) the at least one part incubated in the presence of the test compound and
   (ii) in the at least one part incubated in the absence of the test compound;
(e) determining selectivity of the test compound to induce the phenotype referred to in (d) in the one sub population referred to in step (d) over all other subpopulations by dividing (i) through (ii) wherein the test compound selectively induces the phenotype referred to in (d) if (i) divided through (ii) is greater than 1, preferably greater than 1.05, 1.1, 1.5, 2, 3 most preferably 5, and selectively inhibits or reduces the phenotype referred to in (d) if it is less than 1, preferably less than 0.95, 0.9, 0.7, 0.5, 0.3, most preferably less than 0.2.

Unlike methods practiced in the art for determining the selectivity of a test compound to induce or inhibit a phenotype in one distinguishable cell population over other cell populations in a total population of cells, the methods of the invention do not require the measurement of absolute cell numbers but rely on the measurement of the fraction of cells with a desired phenotype of a total cell population exhibiting the same phenotype. As a consequence, the methods of the present invention are robust towards variations in the exact cell numbers seeded into assay plates that may vary significantly in particular when automated cell dispensing machinery is used. The methods of the present invention are further robust towards cell loss during subsequent staining and washing steps where cells may be lost differentially from different assay wells. The methods further do not require to be benchmarked against bead standards which introduces another source of error and represents additional effort. The methods of the present invention are thus internally controlled. Also, the methods of the present invention can obtain selectivity information with as few as one concentration point of the compound measured whereas competing methods require multiple concentration points are measured; see e.g. Example 8.

Further, the methods of the invention unlike methods practiced in the art do not require the separation of cell populations comprising a total population of cells prior to determining the selectivity of the test compound with respect to the cell populations comprising the total population of cells. Thus, the methods of the invention do not only rely on the effect of a test compound on an isolated target cell population in order to determine its selectivity, but also take into account the interplay of cells comprised in a complex population of cells. Therefore, the methods of the invention surprisingly allow for the measurement of test compound, selectivity in mixtures of cells whereby the resulting selectivity is inherently robust towards variations in cell numbers between different measurements, is internally controlled and takes into account the interplay between different cell populations comprised in a total population of cells.

Further, the methods of the present invention do not depend on determining an EC50 from a dose-response curve. This is in contrast to the methods in the prior art that require the determination of an EC50 to measure selectivity. The EC50 is the concentration of a compound at which the, half maximum effect induced by the compound or inhibited by the test compound is obtained. The EC50 is often also called IC50 (“inhibitory concentration 50%”) or GI50 (“growth inhibition 50%”) depending on the context. Alternatively, also concentrations at which other percentages of the effect is achieved or inhibited are sometimes used (e.g., EC90, EC80 etc.). The EC50 is typically obtained by measuring response of a cell towards a test compound at 4 or more concentrations and fitting a suitable sigmoidal curve to the data. To determine selectivity of a compound to affect one cell population over another, the EC50 of a compound to affect both cell populations has to be measured independently and the difference in log EC50 is taken as a measure of selectivity. For the purpose of determining an EC50, a measurement that is directly proportional to response to a test compound such as the number of cells with a particular phenotype or the magnitude of an effect are required. Such measurements are inherently sensitive in variation of absolute cell numbers incubated with a test compound prior to measurement of a desired phenotype. Rather, the methods of the present invention rely on fractions of cells of one population exhibiting a particular phenotype of the total number of cells with the same phenotype to determine selectivity. Therefore, the methods of the invention do not rely on the quantification of absolute cell numbers and are internally controlled and allow quantification of selectivity of test compounds by measuring response to test compounds at fewer concentration points than required by methods in the art that require the determination of an EC50 and therefore a full dose-response curve (Example 1). The latter is particularly advantageous when the amount of sample provided is limited as it is often the case with primary patient samples.

EC50 values, which lie at the heart of determining selectivity in methods practiced in the care, cannot be determined from fractions of a subset of cells exhibiting a phenotype of the total number of cells exhibiting the desired phenotype, as illustrated in Example 12. It is therefore not obvious to a person skilled in the art to use the fraction of cells exhibiting a particular phenotype of the total number of cells exhibiting the same phenotype to derive information about selectivity.

In contrast to methods of the prior art, in the methods of the invention, for the purpose of evaluating selectivity of a test compound to affect one sup-population of cells over another, selectivity of a test compound is determined based on the number of cells of a particular sub population of cells that exhibit a particular phenotype of interest relative to the number of cells in the total population of cells that exhibit the same phenotype. The resulting selectivity is therefore inherently robust towards variations in absolute cell numbers between different measurements, as illustrated in Example 13, is internally controlled and takes into account the interplay between different cell populations comprised in a total population of cells. Further, the selectivity can be determined by measuring response of cells to test compounds at fewer concentration points than required by methods in the art.

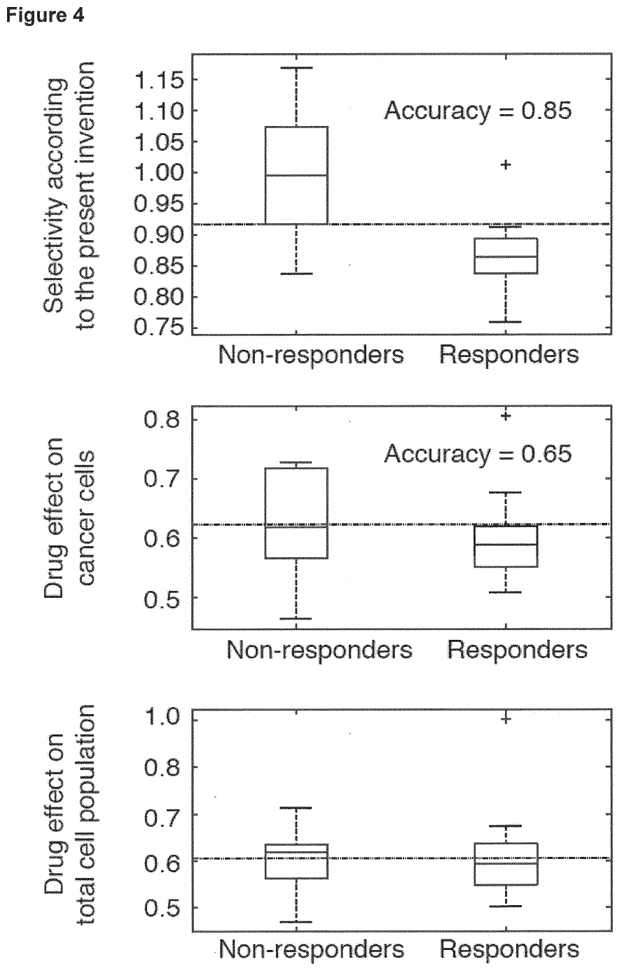

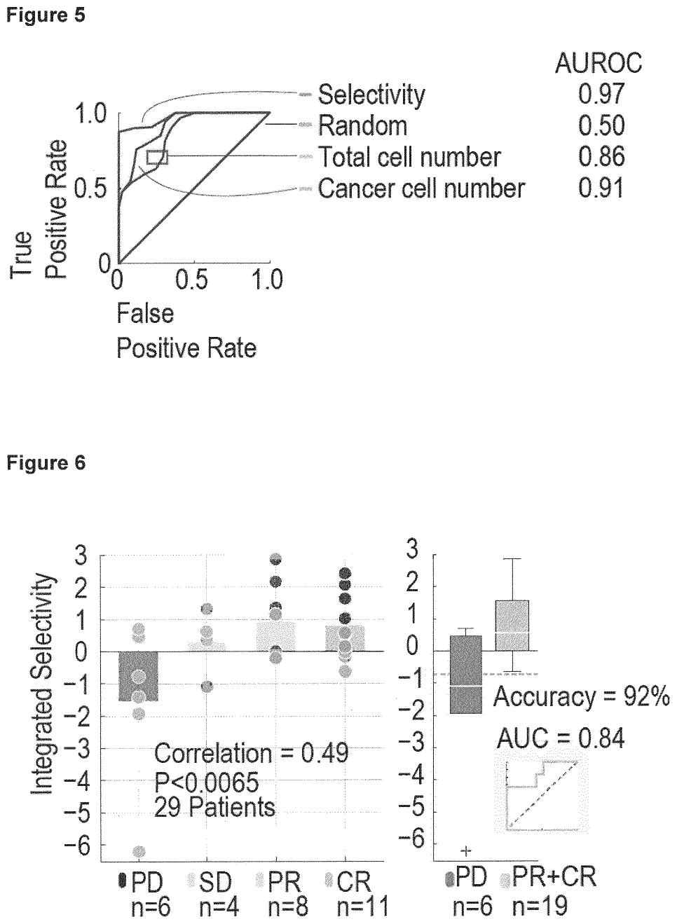

Unlike methods in the prior art for determining whether a subject suffering from cancer will respond or is responsive to treatment with a test compound, the methods of the invention correlate the effect of a test compound on the cancerous populations of cells comprised in a complex population of various cell populations from the patient to the effect of the same test compound on said complex population of cells as a whole. Thus, the methods of the invention not only rely on the effect of a test compound on a target cancer cell population in order to determine its selectivity and make conclusions about whether a patient treated with the test compound will or will not respond, but additionally take into account undesired effects of the test compound on alternative cells comprised in a complex population of cells. In other words, the methods of the present invention quantify the selective ability of an anticancer drug to kill cancerous over non-cancerous cells in order to determine whether a subject suffering from cancer will respond or is responsive to treatment with a test compound or drug. Compared to methods described in the prior art that only measure test compound effect on cancer cells, the methods of the present invention give more accurate information about whether a subject suffering from cancer will respond or is responsive to treatment with a test compound as illustrated in Examples 2-4. Specifically, as shown in the appended examples, the area under the ROC curve (AUROC) value as a measure of the quality by which a method can distinguish two classes using the method of the present invention was 0.97, whereas using a method according to WO 2016/046346 resulted in an AUROC of only 0.91 and basing it on cell number gave an AUROC of 0.86 (see FIG. 5). Similarly, a reduced classification accuracy was observed in Examples 2 and 3 resulting in FIG. 4. As such, the method of the present invention resulted in a classification accuracy of 0.85, whereas if drug response was determined only based on the sensitivity of cancer cells or the sensitivity of the total cell population, a classification accuracy of 0.65 or less was obtained (FIG. 4, middle and bottom panels, respectively). Based on the comparative data provided in the present application, it is thus evident that the method of the present invention provides an improved accuracy.

Further, unlike methods in the prior art for determining whether a subject suffering from cancer will respond or is responsive to treatment with a test compound, the methods of the invention do not rely on the quantification of absolute cell numbers, dose response curves or isolation of cell populations comprising a total population of cells in therefore are more robust towards variation in total cell numbers between different measurements, selective loss of cells from individual measurements, can take into account the interplay of different cell populations that may affect response of a cell population to test compounds (e.g., recognition of a damaged but not dead cancer cell by cells of the immune system) and requires measurement at less concentration points.

The methods of the invention, in step (a), comprise the provision of a sample comprising at least two distinguishable sub-populations of cells in a total population of cells. Within the present invention, the term “distinguishable sub-population” refers to cells that are part of a larger population and can be distinguished from other cells in the population by means of a cell marker. That is, cells of two distinguishable subgroups may belong to the same or different cell type as long as the cells show differing expression profiles of cell markers, which makes them distinguishable using imaging techniques such as confocal microscopy. In order to easily determine whether cells belong to a subgroup of cells, it is preferred to provide cells in form of a monolayer. As shown in the appended Examples, monolayer formation can be done using a method known in the art. For samples comprising only non-adherent cells or mixtures of adherent and non-adherent cells, monolayer formation is done preferably by the method as taught in WO 2016/046346. Accordingly, in a preferred embodiment of the invention, the methods of the invention further comprise, subsequent to step (b) and prior to further analysis, the formation of a monolayer comprising the cells of the cell sample used. In this respect, the type of the cell sample used in the methods of the present invention is not particularly limited as long as it comprises at least two distinguishable sub-populations of cells. In a preferred embodiment of the invention, however, the cell sample used is a PBMC sample or a bone-marrow sample.

Cell markers are proteins expressed by a cell of a particular type that alone or in combination with other proteins allow cells of this type to be distinguished from other cell types. That is, by using cell markers expressed on the surface of cells or within (including within the cytoplasm or within an internal membrane) cells comprised in the total population of cells as used herein, e.g. as comprised in a sample obtained from a donor, can be distinguished and thus attributed to distinguishable sub-populations. Accordingly, the two or more distinguishable subgroups of cells are not limited to cells belonging to different cell types. Rather, cells of the two or more distinguishable subgroups may be of the same cell type as long as the subgroups are distinguishable using cell markers, e.g. those expressed on their surface, e.g. cells of the same cell type at different disease stages, and/or cells of the same type but of different activation states and/or cells of the same type but of different differentiation stages.

Peripheral blood mononuclear cells (PBMCs) are blood cells having a round nucleus (as opposed to a lobed nucleus). PBMCs comprise lymphocytes (B-cells, T-cells (CD4 or CD8 positive), and NK cells), monocytes (dendritic cell and macrophage precursor), macrophages, and dendritic cells. These blood cells are a critical component in the immune system to fight infection and adapt to intruders. In context of some embodiments of the present invention, it is preferred to use ficoll density gradient purified PBMCs, preferably human PBMCs, for creation of the PBMC monolayer of the invention or the cell-culture device comprising the PBMC monolayer or for use in the methods provided in some aspects of the present invention. The present invention can be used with any mononuclear cells, In a preferred embodiment, the invention can determine, but is not limited to determining the selectivity of a test compound with respect to a target cell comprised in a PBMC or bone-marrow cell sample, i.e. selectivity towards cells within the following groups of cells, and cells within the lineage of the cells, including terminal cell states: Hematopoietic stem cells (including, but not limited to, common lymphoid progenitor, common myeloid progenitor, and their maturation lineage and terminal states including pro-B-cell, B-cell, double negative t-cells, positive T-cell, plasma-B-cell, NK-cells, monocytes (macrophage, dendritic cells)). These can be found, but not limited to, within peripheral blood, bone marrow (flat bone localized), cord blood, spleen, thymus, lymph tissue, and any fluid buildup result of a disease such as pleural fluid. Cells may be in any healthy or diseased state.

PBMCs cells for use in the methods described herein can be isolated from whole blood using any suitable method known in the art or described herein. For example, the protocol described by Panda et al. may be used (Panda, S. and Ravindran, B. (2013). Isolation of Human PBMCs. Bio-protocol 3(3): e323). Preferably, density gradient centrifugation is used for isolation. Such density gradient centrifugation separates whole blood into components separated by layers, e.g., a top layer of plasma, followed by a layer of PBMCs and a bottom fraction of polymorphonuclear cells (such as neutrophils and eosinophils) and erythrocytes. The polymorphonuclear cells can be further isolated by lysing the red blood cells, i.e. non-nucleated cells. Common density gradients useful for such centrifugation include, but are not limited to, Ficoll (a hydrophilic polysaccharide, e.g., Ficoll®-Paque (GE Healthcare, Upsalla, Sweden) and SepMate™ (StemCell Technologies, Inc., Köln, Germany).

Bone-marrow cells for use in the methods described herein can be isolated from bone marrow using any suitable method known in the art. In particular, density gradient centrifugation and magnetic beads can be used to separate bone-marrow cells from other components of such samples. For example, MACS cell separation reagents may be used (Miltenyi Biotec, Bergisch Gladbach, Germany).

As is known in the art, such isolated cultures may contain a small percentage of one or more populations of another cell type, e.g., non-nucleated cells such as red blood cells. The PBMCs may be further isolated and/or purified from such other cell populations as is known in the art and/or as described herein; for example, methods of lysing red blood cells are commonly use to remove such cells from the isolated PBMCs. However, the methods of the invention are not reliant on further purification methods, and the isolated PBMCs isolated herein may be directly used. Accordingly, the methods disclosed herein may or may not comprise lysing of red blood cells from within the sample of isolated PBMCs. However, where present, it is believed that the presence of non-nucleated cells, e.g., red blood cells, being generally smaller than PBMCs, settle on the culture surface below and between the PBMCs, and potentially interfere with the formation of a monolayer suitable for imaging. Therefore it is preferred that the concentration of non-nucleated cells, e.g., red blood cells, relative to PMBCs is between about 500 to 1, more preferably about 250 to 1, most preferably about 100 to 1, with the preferential concentration as low as possible. That is, it is most preferred that the isolated PBMC sample used in the methods disclosed herein contains less than about 100 non-nucleated cells, e.g. red blood cells, per PBMC.

Subsequent to providing a sample, in particular a sample comprising at least two distinguishable sub-populations of cells in a total population of cells, as detailed above, and in particular a sample comprising PBMCs or bone-marrow cells, the sample is divided into at least two parts. Alternatively, instead of dividing the sample provided in (a), at least two samples of identical origin and type may be provided, i.e. samples that require no further division. The two parts may have equal size or be of different size. However, it is preferred that each of the at least two parts comprises a similar, preferably identical, ratio of cells of each distinguishable sub-population.

Subsequently to dividing the sample into at least two parts, at least one of the parts is incubated in the absence of a test compound, i.e. the test compound whose selectivity is to be determined. That is, the part is used as control/reference part.

At least one of the remaining parts obtained in step (b) is incubated in presence of the test compound. In this respect, the test compound or the multiple test compounds is/are not particularly limited as long as it/they are generally suitable for use as pharmaceutical. However, it is preferred that said test compound(s) is/are selected from compounds known to be effective in the treatment of a disease, in particular a hematologic malignancy and/or a malignancy of myeloid and/or lymphoid tissue, inflammatory and autoimmune diseases. Compounds known to be effective in the treatment of such diseases comprise chemical compounds and biological compounds, such as, for example, antibodies. Examples of compounds known to be effective in the treatment of such diseases include but are not limited to Alemtuzumab, Anagrelide, Arsenic trioxide, Asparaginase, ATRA, Azacitidine, Bendamustin, Blinatumomab, Bortezomib, Bosutinib, Brentuximab vedotin, Busulfan, Ceplene, Chlorambucil, Cladribine, Clofarabine, Cyclophosphamide, Cytarabine, Dasatinib, Daunorubicin, Decitabine, Denileukin diftitox, Dexamethasone, Doxorubicin, Duvelisib, EGCG=Epigallocatechin gallate, Etoposide, Filgrastim, Fludarabine, Gemtuzumab ozogamicin, histamine dihydrochloride, Homoharringtonine, Hydroxyurea, Ibrutinib, Idarubicin, ldelalisib, Ifosfamide, Imatinib, Interferon Alfa-2a, Recombinant, Interferon Alfa-2b, Recombinant, Intravenous Immunoglobulin, L-asparaginase, Lenalidomide, Masitinib, Melphalan, Mercaptopurine, Methotrexate, Midostaurin, Mitoxantrone, MK-3475=Pembrolizumab, Nilotinib, Pegaspargase, Peginterferon alfa-2a, Plerixafor, Ponatinib, Prednisolone, Prednisone, R115777, RAD001 (Everolimus), Rituximab, Ruxolotinib, Selinexor (KPT-330), Sorafenib, Sunitinib, Thalidomide, Topotecan, Tretinoin, Vinblastine, Vincristine, Vorinostat, Zoledronate, ABL001, ABT-199=Venetoclax, ABT-263=Navitoclax, ABT-510, ABT-737, ABT-869=Linifanib, AC220=Quizartinib, AE-941=Neovastat, AG-858, AGRO100, Aminopterin, Asparaginase Erwinia chrysanthemi, AT7519, AT9283, AVN-944, Bafetinib, Bectumomab, Bestatin, beta alethine, Bexarotene, BEZ235, BI 2536, Buparlisib (BKM120), Carfilzomib, Carmustine, Ceritinib, CGC-11047, CHIR-258, CHR-2797, CMC-544=Inotuzumab ozogamicin, CMLVAX100, CNF1010, CP-4055, Crenolanib, Crizotinib, Ellagic Acid, Elsamitrucin, Epoetin Zeta, Epratuzumab, FAV-201, Favld, Flavopiridol, G4544, Galiximab, gallium maltolate, Gallium nitrate, Givinostat, GMX1777, GP1-0100, Grn163I, GTI 2040, IDM-4, Interferon alfacon-1, IPH 1101, ISS-1018, Ixabepilone, JQ1, Lestaurtinib, Mechlorethamine, MEDI4736, MGCD-0103, MLN-518=Tandutinib, motexafin gadolinium, Natural alpha interferon, Nelarabine, Obatoclax, Obinutuzumab, OSI-461, Panobinostat, PF-114, PI-88, Pivaloyloxymethyl butyrate, Pixantrone, Pomalidomide, PPI-2458, Pralatrexate, Proleukin, PU-H71, Ranolazine, Rebastinib, Samarium (153sm) lexidronam, SGN-30, Skeletal targeted radiotherapy, Tacedinaline, Tamibarotene, Temsirolimus, Tioguanine, Troxacitabine, Vindesine, VNP 40101M, Volasertib, XL228, hydroxychloroquine (Plaquenil), leflunomide (Arava), methotrexate (Trexall), sulfasalazine (Azulfidine), minocycline (Minocin), abatacept (Orencia), rituximab (Rituxan), tocilizumab (Actemra), anakinra (Kineret), adalimumab (Humira), etanercept (Enbrel), infliximab (Remicade), certolizumab, pegol (Cimzia), golimumab (Simponi), tofacitinib (Xeljanz, Xeljanz XR), baricitinib, celecoxib (Celebrex), ibuprofen (prescription-strength), nabumetone (Relafen), naproxen sodium (Anaprox), naproxen (Naprosyn), piroxicam (Feldene), diclofenac (Voltaren, Diclofenac Sodium XR, Cataflam, Cambia), diflunisal, indomethacin (Indocin), ketoprofen (Orudis, Ketoprofen ER, Oruvail, Actron), etodolac (Lodine), fenoprofen (Nalfon), flurbiprofen, ketorolac (Toradol), meclofenamate, mefenamic acid (Ponstel), meloxicam (Mobic), oxaprozin (Daypro), sulindac (Clinoril), salsalate (Disalcid, Amigesic, Marthritic, Salflex, Mono-Gesic, Anaflex, Salsitab), tolmetin (Tolectin), betamethasone, prednisone (Deltasone, Sterapred, Liquid Pred), dexamethasone (Dexpak, Taperpak, Decadron, Hexadrol), cortisone, hydrocortisone (Cortef, A-Hydrocort), methylprednisolone (Medrol, Methacort, Depopred, Predacorten), prednisolone, cyclophosphamide (Cytoxan), cyclosporine (Gengraf, Neoral, Sandimmune), azathioprine (Azasan, Imuran), and hydroxychloroquine (Plaquenil).

Accordingly, in the methods of the present invention, at least one part of the cell sample is incubated in the absence of the test compound(s) and at least one sample is incubated in the presence of the test compound(s).

In this respect, in some embodiments of the methods of the invention, cells, in particular PBMCs, are incubated subsequently to isolation at a density of about 100 cells per mm² growth area to about 30000 cells per mm² growth area. Preferably, the cells, in particular PBMCs, are incubated at a density of about 500 cells per mm² growth area to about 20000 cells per mm² growth area, about 1000 cells per mm² growth area to about 10000 cells per mm² growth area, about 1000 cells per mm² growth area to about 5000 cells per mm² growth area, or about 1000 cells per mm² growth area to about 3000 cells per mm² growth area. Most preferably the cells, in particular PBMCs, are incubated at a density of about 2000 cells per mm² growth area. The term “about” shall have the meaning of within 10%, more preferably within 5%, of a given value or range. Accordingly, the cells, in particular PBMCs, are, in some embodiments, incubated in the methods of the invention to have in the culture device a density of about 100, i.e. from 90 to 110, cells per mm² growth area to about 30000, i.e. 27000 to 33000, cells per mm² growth area. More preferably, the cells, in particular PBMCs, are incubated at a density of about 500, i.e. 450 to 550, cells per mm² growth area to about 20000, i.e. 18000 to 22000, cells per mm² growth area, about 1000, i.e. 900 to 1100, cells per mm² growth area to about 10000, i.e. 9000 to 11000, cells per mm² growth area, about 1000, i.e. 900 to 1100, cells per mm² growth area to about 5000, i.e. 4500 to 5500, cells per mm² growth area, or about 1000, i.e. 900 to 1100, cells per mm² growth area to about 3000, i.e. 2700 to 3300, cells per mm² growth area. Most preferably the cells, in particular PBMCs, are incubated at a density of about 2000, i.e. 1800 to 2200, cells per mm² growth area.

The number of cells, in particular PBMCs, can be determined using standard methods known in the art. In particular, the number of PBMCs can be determined by cell counting using a hemocytometer or the method described by Chan et al. (Chan et al. (2013) J. Immunol. Methods 388 (1-2), 25-32). The number of bone-marrow cells can also be determined using methods well known in the art. In particular, bone-marrow cells can be determined using cell counting. Other cells may also be counted using methods well-known in the art.

Incubation is carried out in a culture medium. A person skilled in the art is well aware of suitable methods to maintain viability of cells, in particular PBMCs or bone-marrow cells. However, the culture medium to be used in the methods of the invention is not particularly limited. In this regard, medium stands for liquids with nutrients and substances necessary for cultivation of cells. Liquid culture media for culturing eucaryotic cells are known to the person skilled in the art (e.g., DMEM, RPMI 1640, etc). Suitable media may be selected depending on the type of cells to be cultured. For example, PBMCs or bone-marrow cells may be cultivated in RPMI 1640 10% FCS. Any suitable media may be chosen, however, media components should be selected that are known to not artificially influence PBMC response and/or bone-marrow cell response. Supplements describe substances to be added to culture media in order to induce or modify cell function (e.g. cytokines, growth and differentiation factors, mitogens, serum). Supplements are known to the person of skill in the art. One example of a serum commonly used with eukaryotic cells is fetal calf serum. The culture media may further be supplemented with antibiotics, such as penicillin, streptomycin, ciprofloxacin etc. In one embodiment, test substances and/or stimulatory agents may be added to living cell material in each individual unit separately. Test substances may be pharmaceutical drugs or drug components. Stimulators may comprise any of the substances which support maintenance, growth or differentiation of cells. In a particular embodiment, stimulators are substances which act on immune cells, e.g. by activation of immune cells. Stimulators for activation of immune cells are known from the prior art. Such agents may be polypeptides, peptides or antibodies and other stimulators. For example, OKT-3, interferon-alpha, interferon-beta and interferon-gamma, oligoCPGs, mitogens (e.g. PWM, PHA, LPS), etc. Test substances and stimulators may be injected into the cell culture medium. Preferably, PBMCs are cultured in RPMI supplemented with FBS/FCS at 10% (preferably but not necessarily having low endotoxin raitings to minimize activation). PBMC cultures may furthermore comprise human serum from the PBMC donor.

The term “growth area” as used within the meaning of the invention refers to the surface within a culture device upon which cells rest. The “density” as used within the meaning of the invention is the quantity of cells per unit area of the surface within the device upon which the cells rest. The culture device may be produced of any material compatible with cell culture, in particular, non-cytotoxic cell culture tested material. Examples for the material are plastic materials, e.g., thermoplastic or duroplastic materials. Examples of suitable plastics are polyethylene, polypropylene, polysulfone, polycarbonate/polyetherethylketone (PEEK) or polytetrafluorethylene (PTFE). In particular, the device is suitable for the culture and/or maintenance of PBMCs. Typical culture devices known in the art and of use in the invention include culture flasks, dishes, plates, and multi-well plates. Of particular use are multi-well plates, which provide the ability to separately maintain multiple cultures, e.g., for multiple perturbations, with minimal material requirements, e.g., minimal media requirements. Preferred culture devices include 96 well plates, 384 well plates and 1536 well plates. As known in the art in connection with imaging analysis of cultures, in particular, fluorescence imaging, it is particularly preferred to use black wall plates specifically designed for imaging that reduce background fluorescence/background optical interference with minimal light scatter and reduced crosstalk. The culture device may be sterilized. In a most preferred embodiment, a multiwell imaging plate is used, the plate including multiple wells, wherein at least some of the wells comprise a first chamber, the first chamber being formed by one or more first sidewalls and a bottom wall; a second chamber, the second chamber being formed by one or more second sidewalls and including an opening for introducing liquids, wherein the second chamber is arranged on top of the first chamber; an intermediate floor provided between the first chamber and the second chamber which forms a disturbance blocking structure; wherein the intermediate floor is provided with at least one through hole that provides a liquid connection between the first and second chambers; wherein the through hole is configured for a tip of a pipette being inserted through the second chamber into the first chamber through said through hole.

The device is in particular of use in automated imaging systems and analysis. Thus, it is preferred that the device/culture device is suitable for use in such systems. In a non-limiting example, the culture device may be translucent. Culture dishes and plates of use for imaging, e.g., fluorescent imaging, are well known in the art and are commercially available. A non-limiting example of a commercially available culture plate for use in the practice of the invention is Corning® 384-well, tissue-culture treated black lid, clear bottom plates (Corning Inc., Massachusetts, USA) or Corning® 384 Well Flat Clear Bottom Black Polystyrene TC-Treated Microplates (Product #3712). Another example is the Perkin Elmer Cellcarrier®.

As outlined above, one surprising technical advantage of the methods of the invention is that the methods determine/rely on selectivity of a test compound towards target cells that is determined in an environment that more closely represents the natural environment of the target cells within a complex population of cells. That is, in the methods of the present invention, naturally-occurring cell-cell interactions are preferably maintained. In this respect, the skilled person is aware of means and methods how to determine/assess/track/verify cell-cell interactions. In particular, the person skilled in the art can distinguish between natural-occurring cell-cell interactions and those introduced during the preparation of a cell sample. As such, the skilled person understands that cells of the same type and/or cells of different types interact in a living organism. Moreover, the person skilled in the art understands that cells of distinguishable subgroups of cells comprised in the total population of cells interact in a living organism. In the present invention, it is preferred that the majority of cells comprised in the cell sample maintain their natural-occurring cell-cell interactions. That is, the majority of cells, in particular at least 50% of the cells comprised in the cell sample, preferably 60%, 65%, 70%, 75%, 80%, 85%, 90%, 95%, or 100% of the cells comprised in the cell sample interact with the same cell or a cell of the same cell type or a cell of the same distinguishable subgroup as in vivo. Cell-cell interactions can be verified/assessed/determined using methods well-known in the art. For example, confocal microscopy can be used to assess/determine/verify whether cell-cell interactions are between cells interacting in a natural environment or between cells that do not show interaction in a natural environment. Such non-natural cell-cell interactions may be due to, inter alia, cell clumping.

Moreover, in the methods of the present invention, the majority of cells is preferably found in a physiologically-relevant state, which means that preferably 60%, 70%, 80%, 90%, 95% or 100% of the cells are in a physiologically-relevant state. The above percentages of cells in a physiologically-relevant state used in the methods of the present invention are determined/measured/assessed using methods well-known in the art. In particular, whether a cell sample comprises cells found in a physiologically-relevant state is determined by quantification of cells comprised in the cell sample. This may be done using methods well known in the art. In particular, quantification may be done through image analysis compared to the cells in a reference sample, for example in peripheral blood or bone marrow of a reference individual or multiple reference individuals, e.g. one or more healthy donor(s) where the cell sample, in particular the PBMC or bone-marrow cell sample is derived from a diseased donor. Quantification of cells is a standard diagnostic tool. Thresholds of cell subpopulations comprised in cell samples, in particular PBMCs and/or bone-marrow cells are well documented for healthy donors and diseased donors. Accordingly, based on differences in samples to be assessed using the means and methods of the present invention, the physiological-relevance can be determined. Documentation of cell subpopulations comprised in hematopoietic cells can be found, for example, in Hallek et al. (2008) Blood 111(12). Accordingly, quantification and further means and methods, for example determination of cell-cell interactions using microscopy, allow the determination whether a cell sample represents a physiological-relevant state.

The inventors have provided methods that allow analysis of samples comprising cells which maintain naturally-occurring cell-cell interactions and which are mainly found in a physiologically relevant state. Thus, in a preferred embodiment of the invention, the cells are analyzed in the form of monolayers. In this respect, it has been found by the inventors that incubation at the cell densities disclosed above, results in formation of an imageable monolayer of cells, in particular PBMCs or bone-marrow cells. The monolayers are formed subsequent to dividing the sample into at least two parts, i.e. step (b) and prior to further analysis. Thus, the methods of the present invention allow, inter alfa, imaging and/or microscopic analysis of cell population, in particular PBMC populations and/or bone-marrow populations. Monolayer as used herein implies a single layer of cells found predominantly within the same focal plane of the imaging device, e.g., microscope or automated camera as is known in the art or described herein. The term single layer is used to mean that the cells within this layer form a culture that is predominantly 2-dimensional, i.e., the culture is predominantly a layer of single cells. That is, within the culture, the majority of the cells are not found resting on or above other cells, and are not found in aggregates (e.g., consisting of groups of cells that extend above the layer of single cells by comprising cells that rest on or above other cells). Thus, the cell monolayer, in particular PBMC monolayer within the meaning of the invention preferably comprises a horizontal layer of cells, in particular PBMC cells having a thickness of the height of one single cell, in particular PBMC. Likewise, the bone-marrow cell monolayer within the meaning of the invention preferably comprises a horizontal layer of bone-marrow cells having a thickness of the height of one single bone-marrow cell. As used herein, the term monolayer does not exclude that within the culture vessel cell aggregates or multilayer constructs (i.e., areas having cell cultures with a height of greater than one cell; in particular PBMC cell or one bone-marrow cell, respectively) or areas without cells may be found. Rather, the term is used to mean that the cultures of the invention will have the majority of their imageable or visible area (e.g., by microscopic methods) consisting of a single layer of cells. This is most easily accomplished as providing a single layer of cells on a cell culture surface. However, as the skilled person will appreciate, other formats of cell samples may also be used in the methods of the present invention.

In the case of non-adherent cells that are to be used, for example PBMCs or bone marrow cells, it is known that such cells typically do not form strong contacts with cell-culture surfaces or strong cell-to-cell contacts. Therefore, the cell monolayers used in the present invention, in particular the PBMC monolayers used in various aspects of the present invention are not envisioned to be necessarily equivalent to monolayers of adherent cells as understood in the art, i.e., comprising a layer of cells firmly attached, evenly spread, and covering the majority of the culture surface. Rather, in some embodiments, the cell monolayer, in particular the PBMC monolayer used in the invention may comprise cultures of high density comprising a majority of cells in direct contact with one or more other cells, but not necessarily adhered to the culture surface, or may comprise cultures of low density, wherein cells are within the monolayer but have no (direct physical) contact with any other cell in the culture. The cell monolayers used in certain aspects of the present invention may also comprise cultures of intermediate density, having discrete areas wherein cells are in contact with one or more cells and other areas where the cells exhibit no contact with other cells.

The cells, in particular PBMCs, bone marrow cells, or other adherent- and non-adherent primary cells for use in the methods of the present invention may be isolated from a sample obtained from a healthy subject, i.e. not suspected to suffer from a disease or suspected to be predisposed to a disease, or may be isolated from a sample obtained from a subject known to be suffering from a disease or suspected to suffer from a disease. The diagnosis of the disease state of the subject may be made by standard methods routinely performed by those skilled in the art, e.g., physicians. Such traditional methods may be supplemented or replaced with the methods of the present invention. For example, in order to determine whether a subject suffers or is likely to suffer from a disease, a cell-cell interaction pattern is determined that is characteristic for the respective disease using samples from subjects known to suffer from the disease. Additionally, or alternatively, the cell-cell interaction pattern of a healthy donor may be used to determine differences that likely are due to the respective disease. Cell interaction pattern here refers to the propensity of one or more different cell types or cell populations to interact with each other determined according to the present invention.

Subsequent to incubation, the number of cells in one of the at least two sub-populations that exhibit a distinguishable phenotype is determined, relative to the number of cells in the total population of cells that exhibit the same phenotype in (i) the at least one part incubated in the presence of the test compound and (ii) in the at least one part incubated in the absence of the test compound. The skilled person is aware of various methods available to count cells of a particular phenotype. In this respect, “phenotype” refers to an observable characteristic or trait of a cell. The phenotype results from the expression of an organism's genetic code, its genotype, as well as the influence of environmental factors and the interactions between the two. Phenotypes of cells include but are not limited to a particular cell morphology, size, both of the cell as a whole or of subcellular parts, cellular viability, expression of a protein, location of a particular protein in a particular location, co-localization of proteins, post-translational modifications of proteins such as phosphorylation, nutrient uptake and consumtpion and others. A phenotype may also be functional in nature in that a particular trait is only exhibited in response to a certain stimulus such as stimulation with a cytokine, a pathogen or some other extracellular or intracellular stimulus.

In a preferred embodiment of the invention, the distinguishable phenotype in step (d) is viability and (i) if the selectivity determined in step (e) is <1 the test compound is determined to selectively reduce the number of viable cells of the one sub-population of step (d), and (ii) if the selectivity determined in step (e) is >1 the test compound is determined to selectively improve viability of the one sub-population and/or to selectively reduce the viability of one or more of the sub-population(s) other than the one sub-population of step (d).

Subsequently, selectivity of the test compound(s) to induce the phenotype of the cells counted in the previous step as part of one of the sub-populations is determined. In the present invention, this is done by dividing (i), as immediately above, through (ii), as immediately above, wherein the test compound selectively induces the phenotype referred to in (d) if (i) divided through (ii) is greater than 1, preferably greater than 1.05, 1.1, 1.5, 2, 3 most preferably 5, and selectively inhibits or reduces the phenotype referred to in (d) if it is less than 1, preferably less than 0.95, 0.9, 0.7, 0.5, 0.3, most preferably less than 0.2.

As shown in the appended Examples, the resulting selectivity of the test compound(s) towards a target cell population is more robust towards variation of total cell numbers in different measurements carried out in the course of the method, requires measurement at fewer concentrations and takes into account the interplay of different cell populations comprising a total population of cells.

In a further embodiment of the invention, a method for determining whether a subject suffering from cancer will respond or is responsive to treatment with a test compound is provided, wherein the method comprises the steps (a) providing a sample obtained from the subject comprising at least two sub-populations of cells in a total population cells, wherein at least one sub-population corresponds to cancerous cells and at least one sub-population corresponds to non-cancerous cells; (b) dividing the sample into at least two parts; (c) incubating at least one part obtained in step (b) in the absence of a test compound and at least one part in the presence of a test compound; (d) determining the number of viable cells in at least one of the sub-populations corresponding to cancer cells relative to the number of viable cells in the total population of cells in (i) the at least one part incubated in the presence of the test compound and (ii) the at least one part incubated in the absence of the test compound; and (e) determining whether the subject will respond or is responsive to treatment with the test compound by dividing (i) through (ii), wherein the subject will respond or is responsive to treatment if the resulting value is less than 1, preferably less than 0.95, 0.9, 0.8, 0.6, 0.4 most preferably less than 0.2.

Accordingly, the invention provides for methods for use in a diagnostic method for determining whether a subject will respond or is responsive to treatment with a test compound, in particular a therapeutic agent. As detailed above, a given test compound, in particular therapeutic agent, e.g. one of the therapeutic agents listed further above, may exhibit a therapeutic effect different for individual subjects suffering from the same or similar disease, e.g. cancer. Therefore, it is advantageous to determine the selectivity of a test compound, in particular therapeutic agent, individually for a given subject. In the above method of the invention, selectivity of a test compound, in particular a therapeutic agent, may be determined in a highly reliable manner with an improved probability as compared to methods of the prior art that a test compound, in particular therapeutic agent, that was determined to have an improved selectivity as compared to one or more alternative test compound(s), in particular therapeutic agent(s), will exhibit an advantageous effect in an in vivo context.

That is, analysis of the selectivity, using the methods provided herein, of a test compound towards a sub-population of cells within the sample/image is predictive of the response of the disease state to the therapy tested in the donor; in this respect the methods of the invention provide advantages over current methods available in the art.

In one embodiment, the method for determining whether a subject suffering from cancer will respond or is responsive to treatment with a test compound is repeated for at least two test compounds and whether the subject will respond or is responsive to treatment with a combination of the at least two test compounds is determined by subtracting the values obtained in (e) for each of the at least two test compounds from 1.0, and summing over the resulting values for the at least two test compounds wherein if the resulting sum is greater than −1, preferably greater than −0.5, 0, 0.5, most preferably greater than 1, the subject is determined to respond or be responsive to treatment with the combination of the at least two test compounds.

In one embodiment of the invention, the test compound used in the methods of the invention may comprise more than one chemical substance. That is, the present invention, in one embodiment, relates to the methods of the invention as disclosed herein, wherein the test compound(s) comprise(s) one or more chemical substances. The presence of more than one chemical substance in the test compound used in the methods of the present invention may provide useful information with respect to, for example, synergistic effects between the at least two chemical substances. That is, chemical substances may influence each other with respect to their selectivity for a given target cell. In order to reliably determine said selectivity and/or in order to use the determined selectivity in the methods of the present invention, it is thus advantageous to use test compound(s) comprising more than one chemical substance.

In a further embodiment of the present invention, the at least one part obtained in step (b) of the methods of the present invention is further divided into at least two parts, wherein each of the at least two parts is incubated with the test compound at different concentrations. Determining the selectivity of a test compound at different concentrations of the test compound and/or determining whether a subject suffering from cancer will respond or is responsive to treatment, with a test compound at different concentrations of the test compound may provide further improved results, because effective concentrations in vivo may vary depending on dosage and timing of an administered test compound. That is, in order to take potential effects associated with the concentration of the test compound into account, the test compound(s) may, in one embodiment of the invention, incubated with the at least two parts obtained in step (b) of the methods of the invention at different concentrations. The skilled person is aware of typical concentrations used in methods known in the art to determine selectivity of a test compound. That is, concentrations will typically be between 100 μM and 100 μM, preferably concentrations of 10 μM and 1 μM, more preferably of 10 μM, 1 ρM and 100 nM are used.

In a preferred embodiment of the invention, in particular in the methods of the present invention where a test compound is incubated at different concentrations with one or more part(s) obtained in step (b) of the methods of the invention, an average selectivity can be calculated in step (e) and used for determining the final selectivity. The average selectivity may provide a more reliable measure that is even further improved over selectivity determined by methods known in the art.

Similarly, in one embodiment of the invention, the methods of the invention comprise in step (c) that at least two parts are incubated in the absence of a test compound and/or at least two parts are incubated in the presence of a test compound, and that in step (d) the average number of cells in the one of the at least two sub-populations relative to the number of cells in the total population of cells in (i) the at least two parts incubated in the presence of the test compound and/or (ii) in the at least two parts incubated in the absence of the test compound is determined. Averaging over multiple measurements reduces potential unwanted effects that may. occur in a single measurement due to natural sample variation. That is, averaging may significantly reduce the expected error of obtained results and thus result in a more reliable outcome of the methods of the invention.

Accordingly, in one embodiment, the invention relates to a method according to the invention, wherein at least one part obtained in step (b) is further divided into at least two parts, wherein each of the at least two parts is incubated in step (c) with the test compound at different concentrations and wherein steps (d) and (e) are repeated for each concentration of the test compound independently to determine a value at each concentration of the test compound whereby an average value over all concentrations is calculated after step (e) and used for determining the final value.

In a further embodiment, the invention relates to a method according to the invention, wherein in step (b) the sample is divided into at least three parts and in step (c) at least two parts are incubated in the absence of a test compound and/or at least two parts are incubated in the presence of a test compound whereby each part incubated in the presence of the test compound is incubated in the presence of the same concentration of the test compound, and wherein in step (d) the number of cells in the one of the at least two sub-populations that exhibit a distinguishable phenotype relative to the number of cells in the total population of cells that exhibit the same distinguishable phenotype is determined for (i) each part incubated in the presence of the test compound independently and/or (ii) each part incubated in the absence of the test compound independently and the average of the relative numbers obtained in (i) and/or the average of the relative numbers obtained in (ii) is used.

In a further embodiment, the invention relates to a method according to the invention, wherein in step (b) the sample is divided into at least three parts and in step (c) at least one part is incubated in the absence of, a test compound and/or at least two parts are incubated in the presence of at least two different concentrations of the test compounds and in step (d) the number of cells in the one Of the at least two sub-populations that exhibit a distinguishable phenotype relative to the number of cells in the total population of cells that exhibit the same distinguishable phenotype is determined for (i) each part incubated in the presence of the test compound independently and/or (ii) each part incubated in the absence of the test compound independently wherein the average of (i) is determined for each concentration independently and/or the average of (ii) is determined and used for further steps and wherein in step (e) the selectivity/value is determined for each concentration of the test compound by dividing the average of (i) for each concentration through the average of (ii) and the final selectivity/value is obtained by averaging the selectivity/value for each concentration.

In a further embodiment, the invention relates to a method according to the invention, wherein the method is repeated for at least two test compounds and the test compound with the lowest value obtained in step (e) is selected for treatment of the subject suffering from cancer.

In a further embodiment, the invention relates to a method according to the invention, wherein the method is repeated for at least three test compounds and the combination of at least two of the at least three test compounds with the highest value obtained by subtracting the values obtained in (e) for each of the at least two test compounds in the combination from 1.0, and summing over the resulting values for the at least two test compounds in the combination, is selected for treatment of the subject suffering from cancer.

Accordingly, the invention also relates to a method for determining which of several test compounds will most likely give the best clinical benefit to a patient suffering from cancer whereby the methods of the invention are repeated for two or more test compounds and the test compound with the lowest value as determined in step (e) of the methods of the invention will be the test compound that most likely gives the patient suffering from cancer the biggest clinical benefit. Thus, the invention also relates to the use of the resulting test compound in the treatment of cancer and the use of the test compound in the manufacture of a pharmaceutical composition for use in treating cancer.

In a further embodiment, the invention relates to a method for determining which of two or more distinct combinations of test compounds comprising two or more test compounds each will most likely give the biggest clinical benefit to a patient suffering from cancer whereby the method according to the invention is repeated for the two or more test compounds comprising the two or more distinct combinations of test compounds each. The combination of the two or more test compounds with the highest resulting sum obtained from subtracting the selectivity values obtained in (e) for each of the at least two test compounds comprising the combination each from 1.0, and summing over the resulting values for the at least two test compounds is the combination of test compounds that will most likely give the patient suffering from cancer the highest clinical benefit. Thus, the invention also relates to the use of the combination of test compounds for treating cancer.

As detailed above, the methods of the present invention may be used for determining selectivity of a test compound towards cells comprised in a total population of cells, wherein the total population of cells comprises at least two distinguishable subgroups of cells. In principle, any population of cells may be used in the methods of the present invention. However, it is preferred to use PBMCs or bone marrow cells. There are various diseases associated with cells comprised in a PBMC or bone marrow cell sample, in particular proliferative diseases such as cancer. Thus, in the methods of the present invention, in particular in the methods for determining whether a subject suffering from cancer will respond or is responsive to treatment with a test compound, the cancer is preferably a cancer associated with PBMCs or bone marrow cells or cells derived from PBMCs or bone marrow cells. The skilled person is aware of cancerous diseases falling within this definition, i.e. types of cancer associated with PBMCs or bone marrow cells or cells derived from PBMCs or bone marrow cells. However, the methods of the present invention are not limited to cancer. That is, the methods of the present invention can be used to determine whether a subject will respond/is responsive to treatment of the following diseases of the following ICD-10 codes (not limited thereto) A00-B99—certain infectious and parasitic diseases; C00-C97 —malignant neoplasms; D70-77—other diseases of the blood forming system; D80-89—certain disorders involving the immune mechanism, not classified elsewhere; D82—Immunodeficiency associated with other major defects; D83—Common variable immunodeficiency; D84—Other immunodeficiency; G35-37—Diseases of the central nervous system; I00-I03—acute rheumatic fever; I05-I09—Chronic rheumatic heart disease; I01—Rheumatic fever with heart involvement; I06—Rheumatic aortic valve diseases; I09—Rheumatic myocarditis; I70—Atherosclerosis; K50—Crohn's disease; K51—Colitis; K52—other noninfective gastroenteritis and colitis; M00-M19 Athropathies; M05—Seropositive rheumatoid arthritis; M06—Other rheumatoid arthritis; M10—Gout; M11—Other crystal athropathies; M35—sicca syndrome; M32—Systemic lupus erythematosus; N70-77—inflammatory diseases of female pelvic organs; P35-39—infections specific to the perinatal period; P50-P61—hemorrhagic and hematological disorders of the fetus and newborn; Z22—Carrier of infectious disease; Z23—Need for immunization against single bacterial diseases; and/or Z24—Need for immunization against certain single viral diseases.

In order to even more reliably determine the selectivity of a test compound and/or determine whether a subject will respond/is responsive to treatment, the methods of the present invention, in a preferred embodiment, make use of a sample that is a tissue sample that contains at least 1% cancerous cells and/or at least 1% non-cancerous cells. More preferably, the tissue sample contains at least 2% cancerous cells and/or at least 2% non-cancerous cells, even more preferably at least 5% cancerous cells and/or at least 5% non-cancerous cells. Most preferably, the tissue samples contain at least 10% cancerous cells and/or at least 10% non-cancerous cells.

As disclosed further above, it is preferred in the methods of the present invention that the sample, in particular the tissue sample, is cultured as a monolayer. In case the sample is derived from cells contained in PBMCs or bone marrow which comprise non adherent cells, the tissue sample is preferably cultured as a non-adherent cell monolayer. The present invention thus provides methods using physiologically relevant, multi-population cell samples, in particular primary hematopoietic samples in imaging studies to determine in a high-throughput manner: 1) the effects of a test compound, for example a test compound to be used in chemotherapy/immunotherapy/immune suppressive therapy on ex vivo cell population diagnostic, or other, markers at a global level based on single-cell analysis, 2) the ability for this technique to provide predictions as to which chemotherapy will be beneficial for which patient based on ex vivo measurements in patient samples, 3) the ability for this technique to determine the effect of many stimuli or a stimulus (e.g. drugs) on the immune function, and 4) for the integration of many patient data sets over time to determine patterns in treatment assessments. In principle, any cell sample may be used in the methods of the present invention such as mononuclear cells from blood, bone marrow, pleural effusion, spleen homogenates, lymph tissue homogenates, skin homogenates. However, it is preferred that mononuclear cells are used. As the skilled person is aware, mononuclear cells samples as used in the methods of the invention comprise, inter alia, PBMCs and bone-marrow cells, as well as others. Accordingly, the cell sample, preferably the monolayer of primary mononuclear cells as used in the methods of the invention may comprise PBMCs and/or bone-marrow cells. That is, while the methods provided herein are described for cells in general or PBMCs, the skilled person understands that methods are provided for bone-marrow cells or further cells. Accordingly, provided herein are methods using bone-marrow cells, methods for determining selectivity of a test compound towards cells comprised in a bone-marrow sample, and methods for determining whether a subject suffering from or predisposed to a disease will respond or is responsive to treatment with a test compound comprising the use of bone-marrow cells.

In this regard, bone marrow is the flexible tissue in the interior of bones. In humans, red blood cells are produced by cores of bone marrow in the heads of long bones in a process known as hematopoiesis. Bone marrow transplants can be conducted to treat severe diseases of the bone marrow, including certain forms of cancer such as leukemia. Additionally, bone marrow stem cells have been successfully transformed into functional neural cells and can also be used to treat illnesses such as inflammatory bowel disease. Accordingly, bone-marrow cells represent a valuable target in the treatment of various diseases, for example cancerous diseases or inflammatory diseases such as inflammatory bowel disease. As such, the methods provided herein using bone-marrow samples obtained from a donor are highly useful in the assessment/determination whether a donor suffers from such a disease or is predisposed to suffer from a disease. In addition, the methods provided herein using bone-marrow cells provide various advantages in high-throughput drug screening and the like.

The dogma of requiring adherent cells (macrophages, HeLa, etc.) to form a stainable and imageable monolayer has been overcome by the provision of monolayers in WO 2016/046346. Prior to the monolayers as described therein, research groups have been unable to implement image-based single cell screening techniques in primary patient samples for high-throughput determination of chemotherapy-induced molecular (biomarker) changes, cancerous blast viability assessments and cell-cell contacts, in particular where the disease state is represented or reflected in non-adherent cells, e.g., in blood-based diseases or conditions such as lymphomas and leukemias. To solve this problem, the inventors of WO 2016/046346 have provided means and methods as well as a methodology and image-analysis pipeline, referenced herein as “pharmacoscopy”, which allows the visualization of adherent and non-adherent cells in a single image, typically requiring only 1/10^{th} of the material needed per perturbation as compared to methods known in the art, and maximizing throughput and speed. Pharmacoscopy can provide the same information gathered by known methods, e.g., flow cytometry, but provides additional advantageous information such as measurement of subcellular phenotypes (protein localization/co-localization) and cellular microenvironment/neighbor relationship. Moreover, the described methods of WO 2016/046346 require fewer cells and therefore less patient material, less liquid volume, and nearly no human intervention; pharmacoscopy thereby greatly increases the number of molecular perturbations which can be tested in parallel and yields more detailed assessments. Moreover, without the need to sort diseased cells from the inherent healthy populations, pharmacoscopy can track drug mediated phenotypic changes while controlling, in parallel, the off-target drug effects. These important controls are done by relating test compound effects on target cells (e.g., cancerous cells) to total cells (e.g. healthy cells) from the same donor, present in the same well, and in the same imaging field. The methods of the present invention use the methodology of WO 2016/046346, but comprise a further surprising and unexpected advantage. In particular, it is now possible to more reliably determine the selectivity of a test compound and/or determine whether a subject suffering from a disease, in particular cancer, will respond or is responsive to treatment with a test compound. This is achieved by taking into account unspecific effects of the test compound on off-target cells comprised in the sample representation, in particular the monolayer.

Using the methods of the invention large numbers of test compounds can be efficiently and quickly analyzed, i.e. their selectivity be determined, using the large numbers of monolayers that may be derived from a single sample obtained from, for example, a patient/subject, in particular a PBMC sample or bone-marrow sample. Typically, the effect of at least 1000, at least 4000, at least 8000, at least 12000, at least 16000, at least 20000, at least 24000, at least 50000, at least 75000, or up to 90000 test compounds or more can be investigated in the multiple monolayers obtained from such a single sample. In certain embodiments, the monolayers provided herein can be imaged and analyzed using multiple channels simultaneously of high content data. The number of channels of data available is dependent only on the particular imaging software and available staining methodologies, which field rapidly advances. Currently available methodologies allow the simultaneous imaging, processing and analysis of at least two channels, and more typically, 4, 5 or 8 channels of high-content data.

In the methods of the present invention, the cells, preferably in the form of monolayers, may be imaged according to any methods known in the art and/or described herein and the methods provided herein may use any imaging technique known in the art. The particular imaging method is not critical and may be decided according to the knowledge of the person of skill in the art. The imaging may or may not require the use of a dye or stain, may comprise imaging of both stained and non-stained components and/or may comprise imaging under conditions wherein the stain is or is not visible (e.g., imaging in bright-field (wherein a fluorescent stain would not be visible) and under UV-lighting (wherein a fluorescent stain would be visible), or combinations thereof. Imaging under bright-field conditions is well known and routine used in the art, and may be performed according to standard methods and/or as described herein. Additionally or alternatively, any other label-free imaging may be used in accordance with the invention. Such label-free methods are known and include, e.g., PhaseFocus imaging (Phase Focus Ltd, Sheffield, UK).

In a preferred embodiment of the invention, the number of viable cancerous and non-cancerous cells is determined using automated microscopy. In an even more preferred embodiment, the number of viable cells is determined as the number of non-fragmented nuclei. Methods to determine the fragmentation of nuclei include but are not limited to staining nuclei with DAPI, or dyes of the Hoechst series of nuclear dyes and assessing their morphology under fluorescence microscopy.

The practice of the invention may also comprise the addition of a detectable label to the cells, preferably the monolayer, in particular the PBMC monolayer (either in connection with label-free methods or independently), which label may be detected using microscopic methods in order to selectively label cells of a particular phenotype such as viability and/or cells of a distinguishable sub-population. The detectable labels may label discrete cellular structures, components or proteins as known in the art. The label may also be attached to antibodies to specifically label and allow the detection of the antibody antigen. In a preferred embodiment, the detectable label allows visualization of the label under visible or ultra-violet light. Thus the detectable label may be fluorescent. A multitude of visual labels are known in the art and are suitable for the invention. The labels may be detectable without further action, or may only become detectable after performance of a secondary step, e.g., addition of a substrate, exposure to enzymatic reactions, or exposure to specific light wavelengths.

Cellular subpopulations, i.e. distinguishable subpopulations as used herein, in particular PBMC subpopulations or bone-marrow cell subpopulations may be identified by detectable labels via expression of one or more markers on the surface of the target cell or inside of the cell. Alternatively or additionally, subpopulations may be defined by the lack of expression of one or more markers on the surface of the target cell or inside the target cell. It may be desirable to test for expression or lack of expression of one or more markers (e.g., two markers, three markers, four markers, etc.) to provide further assurance that a cell expressing or not expressing a marker is in fact a target cell, e.g., a member of desired subpopulation of cells. For example, a “cocktail” of antibodies to different markers may be each coupled (whether directly or indirectly) to the same label or to different labels. As an example, a cocktail of antibodies to different markers may each contain a binding motif that binds the same label (e.g., each may contain an Fc of the same species that is recognized by the same secondary antibody, or each may be biotinylated and specifically bound by the same avidin-coupled label). Optionally, two or more different antibodies or cocktails of antibodies may be utilized. Preferably the cells are stained using at least two labels that can be distinguished from one another, thereby permitting identification of cells that express at least two different markers of the target cell types. Cells may also be stained using at least three, four, five, or more different labels that can be distinguished from one another, thereby permitting detection of cells that express greater numbers of markers of the target cell type. Optionally, a cell may be identified as a cell of the target type if it expresses a preselected number of markers or certain preselected combinations of markers or a cell may be identified as a cell of the target type if it does not express a preselected marker. Additionally, it is not necessary that the marker(s) of the target cell type be unique to the target cells, as long as they permit distinction of the target cells from other cells in the population. In the case of PBMCs, major components of PBMC cell populations are represented by CD11C for dendritic cells, CD14 for macrophages, CD3 (CD4 or CD8 with CD3) for T-cells and CD19 for B-cells. While the foregoing markers overlap on subsets of these major classes of PBMCs, staining with these markers for identifying subpopulations of PBMCs is widely accepted in the field. Further markers suitable for use in methods of the present invention may be found in the CD marker handbook (Becton, Dickinson and Co. 2010, CA, USA). Major cell subpopulations comprised in bone-marrow cells are neutrophilic metamyelocytes, neutrophilic myelocytes, segmented neutrophils, normoblasts and lymphocytes.

It is preferred to use antibodies conjugated to detectable labels in the practice of the invention. Such antibodies allow the targeting of discrete cellular structures and, thus, cocktails of such antibodies (each bearing a different label) may be used to simultaneously visualize multiple targets/cellular structures/cellular components. Care must be taken during staining to avoid monolayer disruption. As the skilled person appreciates, this is particularly problematic with the use of antibody-based labels, as their use normally requires one or more wash-steps to eliminate unbound label that would interfere with accurate visualization, i.e., would result in non-specific staining and/or assay “noise”. Accordingly, the invention encompasses methods for the staining of cell monolayers with a detectable label, in particular, an antibody-based label, which minimizes or eliminates washing requirements subsequent to staining. The methods of the invention may comprise adding the detectable label(s) at concentrations that avoid generation of noise signal in the absence of washing, which can be determined by methods well known in the art and/or described herein. Thus, the invention encompasses the use of labeled antibodies at concentrations above or below that recommended by the antibody manufacturers.

For some exemplary cell populations, cells may only be considered positive for a given marker if that marker exhibits a characteristic localization or pattern within the cell. For instance, a cell may be considered “positive” if a cytoskeletal marker is present in the cytoskeleton and “negative” if there is some diffuse cytoplasmic staining. In such a case, cells may be cultured under suitable conditions (e.g., as adherent cultures) to establish the characteristic localization or pattern within the cell. Suitable culture conditions and time for cytoskeleton assembly (or other processes to establish subcellular organization) that may be necessary for robust detection of a given marker are readily determined by those of ordinary skill in the art. Additionally, markers may readily be chosen which decrease or eliminate the need for adherent culture as a precondition to robust staining.

Dyes useful in labeling proteins are known in the art. In general, a dye is a molecule, compound, or substance that can provide an optically detectable signal, such as a colorimetric, luminescent, bioluminescent, chemiluminescent, phosphorescent, or fluorescent signal. In a preferred embodiment of the invention, the dye is a fluorescent dye. Non-limiting examples of dyes, some of which are commercially available, include CF dyes (Biotium, Inc.), Alexa Fluor dyes (Invitrogen), DyLight dyes (Thermo Fisher), Cy dyes (GE Healthscience), IRDyes (Li-Cor Biosciences, Inc.), and HiLyte dyes (Anaspec, Inc.). In some embodiments, the excitation and/or emission wavelengths of the dye are between 350 nm to 900 nm, or between 400 nm to 700 nm, or between 450-650 nm.

For example, staining may comprise using multiple detectable labels, e.g. antibodies, self-antibodies or patient serum. A stain may be observable under visible light and under ultraviolet light. A stain may comprise an antibody directly or indirectly coupled to a colored reagent or an enzyme capable of producing a colored reagent. When antibodies are used as a component of a stain, a marker can be directly or indirectly coupled to the antibody. Examples of indirect coupling include avidin/biotin coupling, coupling via a secondary antibody, and combinations thereof. For example, cells may be stained with a primary antibody that binds a target-specific antigen, and a secondary antibody that binds the primary antibody or a molecule coupled to the primary antibody can be coupled to a detectable marker. Use of indirect coupling can improve signal to noise ratio, for example by reducing background binding and/or providing signal amplification.

The stain may also comprise a primary or secondary antibody directly or indirectly coupled (as explained above) to a fluorescent label. The fluorescent label may be selected from the group consisting of: Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750 and Alexa Fluor 790, fluoroscein isothiocyanate (FITC), Texas Red, SYBR Green, DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,′-hexachlorofluorescein), Joe (6-carboxy-4′,5′-dichloro-2′,7′-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodamine, Rox (carboxy-X-rhodamine), R6G (Rhodamine 6G), phthalocyanines, azomethines, cyanines (e.g. Cy3, Cy3.5, Cy5), xanthines, succinyifluoresceins, N,N-diethyl-4-(5′-azobenzotriazolyl)-phenylamine, aminoacridine, and quantum dots.

Further exemplary embodiments of the present method utilize antibodies directly or indirectly coupled to a fluorescent molecule, such as ethidium bromide, SYBR Green, fluorescein isothiocyanate (FITC), DyLight Fluors, green fluorescent protein (GFP), TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, TET (6-carboxy-2′,4,7,7′-tetrachlorofluorescein), HEX (6-carboxy-2′,4,4′,5′,7,7′-hexachlorofluorescein), Joe (6-carboxy-4′, 5′-dichloro-2′,7-dimethoxyfluorescein) 5-carboxy-2′,4′,5′,7′-tetrachlorofluorescein, 5-carboxyfluorescein, 5-carboxy rhodamine, Tamra (tetramethylrhodamine), 6-carboxyrhodarnine, Rox (carboxy-X-rhodamine), R6G (Rhodamine 6G), phthalocyanines, azomethines, cyanines (e.g. Cy3, Cy3.5, Cy5), xanthines, succinylfluoresceins, N,N-diethyl-4-(5′-azobenzotriazolyl)-phenylamine and aminoacridine. Other exemplary fluorescent molecules include quantum dots, which are described in the patent literature [see, for example, U.S. Pat. Nos. 6,207,299, 6,322,901, 6,576,291, 6,649,138 (surface modification methods in which mixed hydrophobic/hydrophilic polymer transfer agents are bound to the surface of the quantum dots), U.S. Pat. Nos. 6,682,596, 6,815,064 (for alloyed or mixed shells), each of which patents is incorporated by reference herein)], and in the technical literature [such as “Alternative Routes toward High Quality CdSe Nanocrystals,” (Qu et al., Nano Lett., 1(6); 333-337 (2001)]. Quantum dots having various surface chemistries and fluorescence characteristics are commercially available from Invitrogen Corporation, Eugene, Oreg., Evident Technologies (Troy, N.Y.), and Quantum Dot Corporation (Hayward, Calif.), amongst others. Quantum dot” also includes alloyed quantum dots, such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN. Alloyed quantum dots and methods for making the same are disclosed, for example, in US Application Publication No. 2005/0012182 and PCT Publication WO 2005/001889.

Subsequent to labeling the cells used in the methods of the invention, preferably in the form of a monolayer, the method may further comprise detecting the signal of the detectable label. Depending on the kind of signal emitted by the detectable label, the detection method may be appropriately adapted. It is preferred to use a detection method suitable for detecting fluorescent light emitting labels. The detection method may also be automated according to standard methods known in the art. For example, various computational methods exist that enable a person skilled in the art to analyze and interpret the microscopy images of cells or to establish automated protocols for their analysis. For primary image analysis, including the correction for illumination bias in microscopy images, the identification of individual cells from microscopy images and the measurement of marker intensities and textures as well as nuclear and cellular size and shape and position parameters, the opensource software CellProfiler (e.g. version 2.1.1) can be used. Identification of marker-positive cells (such as CD34+ progenitor cells or viability dye positive cells) can be performed by machine learning using the opensource software CellProfiler Analyst (e.g. version 2.0) and double- or triple-positive cells can be identified by a sequential gating strategy. Plate-overviews for further analysis and hit selection can be created using CellProfiler Analyst as well.

The cellHTS package in Bioconductor (e.g. version 2.14), or Pipeline Pilot (e.g. version 9.0; Accelrys), can both be used for the data analysis subsequent to the primary image analysis, including plate-effect normalization, control-based normalization, and hit selection.

Commercial automated microscopy systems may also be used in the practice of the invention, e.g., PerkinElmer Operetta automated microscope (PerkinElmer Technologies GmbH & Co. KG, Walluf, Germany), which systems may include corresponding image analysis software, e.g., PerkinElmer's Harmony software (e.g. version 3.1.1). Such automated and/or commercial systems can be used to perform primary image analysis, positive cell selection and hit selection from microscopic images according to the methods of the invention.

Subsequent to this primary analysis, the methods of the present invention are performed for determining the selectivity of a test compound towards a cell population having a particular phenotype comprised in a sample comprising at least two distinguishable subpopulations of cells or for determining whether a subject suffering from a disease, in particular cancer, will respond or is responsive to treatment with a test compound, wherein the method comprises the determination of the selectivity of a test compound based on its ability to induce the phenotype above, in particular cell viability.

Based on the result of the method for determining whether a subject suffering from cancer will respond or is responsive to treatment with a test compound of the invention, a treatment decision may be taken, i.e. the test compound determined to have the most advantageous result with respect to whether the subject will respond or is responsive to treatment with the test compound, may be chosen for treatment of the subject.

When calculating the “mean” or “average” of numbers in the methods of the present invention, it is understood that this can refer to the arithmetic mean, the geometric mean and/or related statistical measures that have the aim to estimate a true value of a variable based on repeated measurements associated with a random error. It is further understood by people skilled in the art that in some cases, it may be advantageous to use the median instead of the mean (e.g., in cases where outliers are present but the underlying random variable is normally distributed). In a preferred embodiment, the arithmetic mean is used whenever the methods of the present invention refer to “mean” or “average”.

When a test compound comprises more than one chemical substance, the concentration of the test compound refers to a particular combination of chemical substances at different concentrations and different concentrations of the test compound refer to at least one chemical substance comprising the test compound having a different concentration. A test compound comprising more than one chemical substance having a particular concentration means that all chemical substances comprising the test compound have one particular but not necessarily the same concentration.

“Treatment” or “treating” refers to both therapeutic treatment and prophylactic or preventative measures, wherein the object is to prevent, ameliorate or slow down (lessen) the targeted pathologic condition or disorder, or one or more symptom associated therewith. Similarly, “responsive to” or “responds” and analogous terms refer to indications that the targeted pathological condition, or one or more symptom associated thereof, is prevented, ameliorated or lessened. The terms are also used herein to denote delaying the onset of, inhibiting (e.g. reducing or arresting the growth of), alleviating the effects of, or prolonging, the life of a patient suffering from a disease, in particular a myeloproliferative disease, or indications that such markers have been accomplished. Those in need of treatment include those diagnosed with the disorder, those suspected of having the disorder, those predisposed to have the disorder as well as those in whom the disorder is to be prevented. Hence, the mammal to be treated herein may have been diagnosed as having the disorder or may be predisposed or susceptible to the disorder.

“Response” or “responsive” refers to. a subject showing at least one altered characteristic subsequent to treatment. The altered characteristic of the subject may be amelioration or slowing down of the targeted pathologic condition or disorder.

As used herein, the terms “prevent”, “preventing” and “prevention” refer to the prevention of the occurrence and/or recurrence or onset of one or more symptoms of a cancer disease in a subject resulting from the administration of a prophylactic or therapeutic agent.

The means and methods provided herein are mostly described for primary hematopoietic cells, or all monocyte cells. As the skilled person understands, primary hematopoietic cells comprise, inter alfa, PBMCs and bone-marrow cells. Accordingly, the means and methods provided herein, which are described for PBMCs, are also disclosed for bone-marrow cells, as well as any other mono-nucleated cell.

The test compound(s) used in the methods of the present invention may be therapeutic agent(s) used in the treatment/approved for treatment of a disease, in particular cancer. In this respect, “test compound” within the meaning of the invention are molecules including, without limitation, polypeptides, peptides, glycoproteins, nucleic acids, synthetic and natural drugs, peptoides, polyenes, macrocyles, glycosides, terpenes, terpenoids, aliphatic and aromatic compounds, and their derivatives. In a preferred embodiment, the test compound is a chemical compound such as a synthetic and natural drug. In another preferred embodiment, the test compound effects amelioration and/or cure of a disease, disorder, pathology, and/or the symptoms associated therewith. The polymers may encapsulate one or more test compounds to be used in the methods of the invention.

As detailed immediately above, test compounds may also be selected from known therapeutic agents. In this respect, suitable therapeutic agents include, without limitation, those presented in Goodman and Oilman's The Pharmacological Basis of Therapeutics (e.g., 9th Ed.) or The Merck Index (e.g., 12th Ed.). Genera of therapeutic agents include, without limitation, drugs that influence inflammatory responses, drugs that affect the composition of body fluids, drugs affecting electrolyte metabolism, chemotherapeutic agents (e.g., for hyperproliferative diseases, particularly cancer, for parasitic infections, and for microbial diseases), antineoplastic agents, immunosuppressive agents, drugs affecting the blood and blood-forming organs, hormones and hormone antagonists, vitamins and nutrients, vaccines, oligonucleotides and gene therapies. It will be understood that compositions comprising combinations, e.g. mixtures or blends of two or more active agents, such as two drugs, are also encompassed by the invention.

In one embodiment the therapeutic agent may be a drug or prodrug, antibody or vaccine. The method of the invention may be used to assess whether administration of a therapeutic agent to a patient triggers a response to the therapeutic agent, or a component of a delivery vehicle, excipient, carrier etc. administered with the therapeutic agent.

The precise nature of the therapeutic agent is not limiting to the invention. In non-limiting embodiments the method of the invention may be used to assess response to synthetic small molecules, naturally occurring substances, naturally occurring or synthetically produced biological agents, or any combination of two or more of the foregoing, optionally in combination with excipients, carriers or delivery vehicles.

The viability of cells comprised in the sample to be analyzed, in particular in the monolayers can be determined/assessed/verified using methods well-known in the art. That is, the skilled person is well-aware of methods how to determine/assess/verify the stadium of a cell, for example whether a cell is viable, live, dead or undergoing a process changing its stadium, for example dying as in apoptosis or necrosis. Accordingly, known markers/dyes that specifically recognize/label cells being in a particular stadium can be used in the methods of the invention. That includes dyes/labels that are selective for cells with non-intact membranes or dyes/labels selective for late-stage cell death or early apoptosis. For example, fixable live/dead green can be used (ThermoFisher, catalogue number L-23101), antibodies against cytochrome C, determining DNA turnover or cell proliferation through the use of dyes. Further means and methods how to determine/assess/verify viability of cells comprised in cell sample used in the present invention, in particular in the form of a monolayer are known to the skilled person.

Determining/tracking/assessing/verifying changes of viability and/or cell-cell interactions of the two or more distinguishable subpopulation(s) comprised in the cell sample, in particular the monolayer, in particular PBMC monolayer or bone-marrow cell monolayer, can be done using methods well-known in the art. For example, using microscopy, changes can be determined/tracked/assessed/verified by optical perception. However, for high-throughput applications, it is preferred that an automated method is used, which determines/tracks/assesses/verifies changes of viability and/or cell-cell interactions of individual subpopulations comprised in the monolayers. Such a method comprises identifying subpopulations comprised in the cell sample, preferably the monolayer, e.g. by detectable labels. It can then be determined whether labeled/detected subpopulations show cell-cell interactions, wherein cell-cell interactions may include direct contacts via plasma membranes (as described above) or indirect contacts. Accordingly, a distance parameter, i.e. the threshold defined above, between labeled cells is introduced, which determines the total number of interactions, i.e. how many cell-cell interactions are observed between labeled cells. In this procedure, a labeled cell of a distinguishable subgroup may interact with one or more cells of the second distinguishable subgroup, each interaction being counted. The resulting number is compared to what would be expected by a random distribution function, i.e. by random cell-cell interactions. The interaction propensity can then be calculated using the methods of the invention, i.e. an interaction score, which determines whether interaction is random or directed. Following such a protocol before and after one or more test substance(s) are added to the cell sample of the invention, allows determining/tracking/assessing/verifying changes of cell-cell interactions due to the one or more test compound(s).

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the previous or following description. Also, single alternatives of the embodiments described in the figures and the description and single alternatives of features thereof can be disclaimed from the subject matter of the other aspect of the invention;

Furthermore, in the claims the word “comprising” does not exclude other elements or steps, and the indefinite article “a” or “an” does not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

The patent or application file contains at least one drawing executed in color. Copies of this patent or patent application publication with, color drawing(s) will be provided by the Office upon request and payment of the necessary fee.

The present invention is also illustrated in some aspects by the following figures.

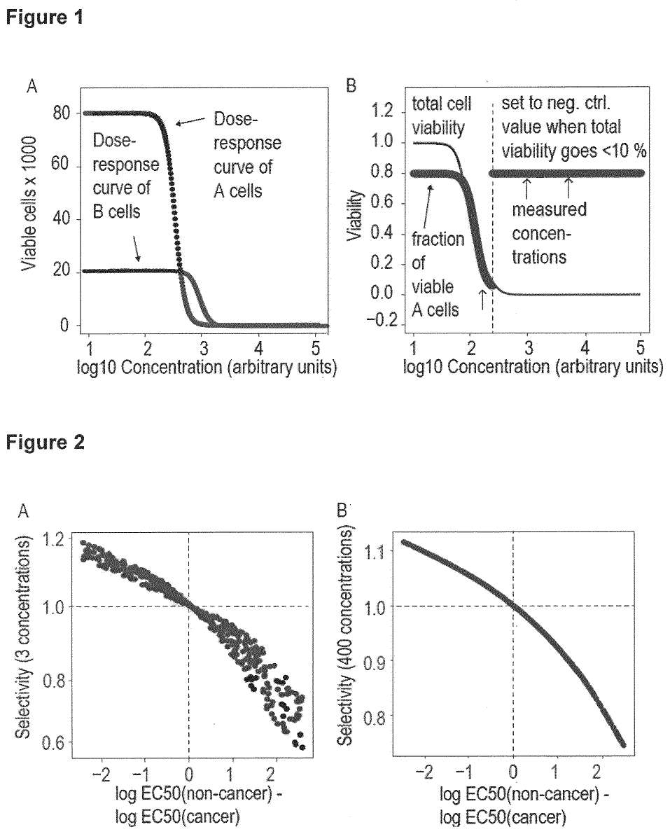

FIG. 1: A. Two hypothetical dose response curves showing viability of cancerous A cells and non-cancerous B cells as a function of drug concentration of a cytotoxic test compound. B. Showing total cell viability and the fraction of viable A cells of viable total cells as a function of test compound concentration. Here, when total cell viability goes below 5% of the starting value, the fraction of viable A cells was set to 0.8 (i.e., the fraction at zero test compound concentration) due to the fact that quantifying small number of cells is associated with large errors. Following this approach, a selectivity of 1 will reliably be assigned to test compounds with strong overall cytotoxicity when following the steps of the methods of the present invention.

FIG. 2: A. Selectivity/value of a cytotoxic test compounds to kill cancer cells determined as described in the present invention as a function of the difference of log EC50 of the test compound against cancer A cells and non-cancerous B cells measuring at three concentration points (c.f., FIG. 1B). B. Selectivity/value determined as described in the present invention as a function of the difference of log EC50 of the test compound towards cancer cells and non-cancerous cells measuring at 400 concentration points. Already measurement at three concentration points is sufficient to obtain selectivity information using the present invention. However, the more concentration points, the more accurate the selectivity/value reflects the difference in log EC50.

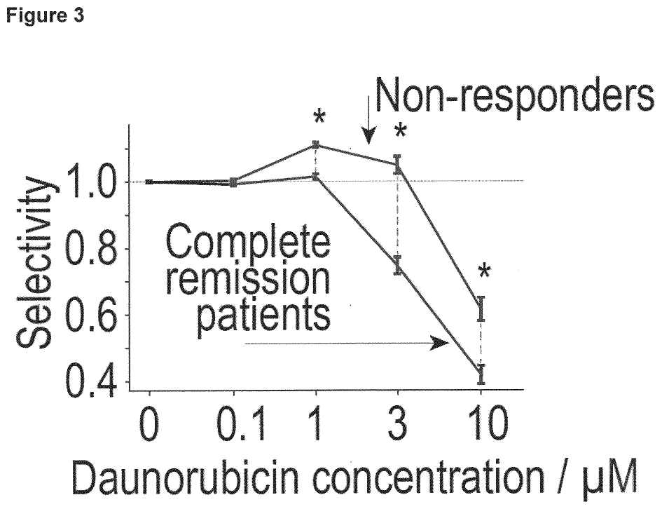

FIG. 3: Selectivities/values of daunorucibin to kill AML blast cells defined as either being CD34+, CD117+ or CD34+/CD117+ determined for individual daunorubicin concentrations using the present invention displayed as a function of daunorucibin concentration. Patients responding to daunorucibin containing therapy had a lower selectivity/value determined using the present invention than patients not responding to daunorubicin based 3+5+7 induction therapy across different daunorubicin concentrations.

FIG. 4: Top panel: Selectivity/value of combinations of daunorubicin+cytarabine+etoposide to kill cancer cells (here defined as CD34 or CD117 expressing cells) in bone marrow samples of AML patients determined according the present invention for responders and non-responders to “3+5+7” induction therapy consisting of the three aforementioned drugs. A cut off of 0.92 allows for classification of patients into responders and non-responders with a total classification accuracy of 0.85. Middle panel: Number of cancerous cells relative to number of cancerous cells at zero drug concentration averaged over all drug concentrations and combinations. This metric only allows classification of patients into responders and non-responders with a total classification accuracy of 0.65. Bottom panel: In analogy to middle panel but based on the total cell number. Accurate classification is not possible using this metric.

FIG. 5: The area under the receiver operator curve was highest (AUROC=0.97) when predicting response of AML patients to daunorubicin containing 3+5+7 induction therapy based on the selectivity of daunorubicin to kill AML blasts determined according to the present invention and using the approach described in Example 4 for downstream data processing. This demonstrates that measuring killing selectivity/value according to the present invention is advantageous compared to e.g., basing the prediction of response on the number of cancerous cells (AUROC=0.91). AML blasts are here defined as in FIG. 2.

FIG. 6: Left: Hematological cancer patients were treated with combinations of 2 or more FDA approved drugs. For each patient the combined selectivity was calculated as the sum of 1 minus the individual selectivites of the drugs given to the patient as determined according to the present invention. The combined selectivity was plot against the response (PD=progressive disease, SD=stable disease, PR=partial remission, CR=complete remission) and correlates with the response. Based on the integrated selectivity, patients could be classified into responders (CR and PR) and non-responders (PD and SD) with 92% accuracy and an AUROC of 0.84.

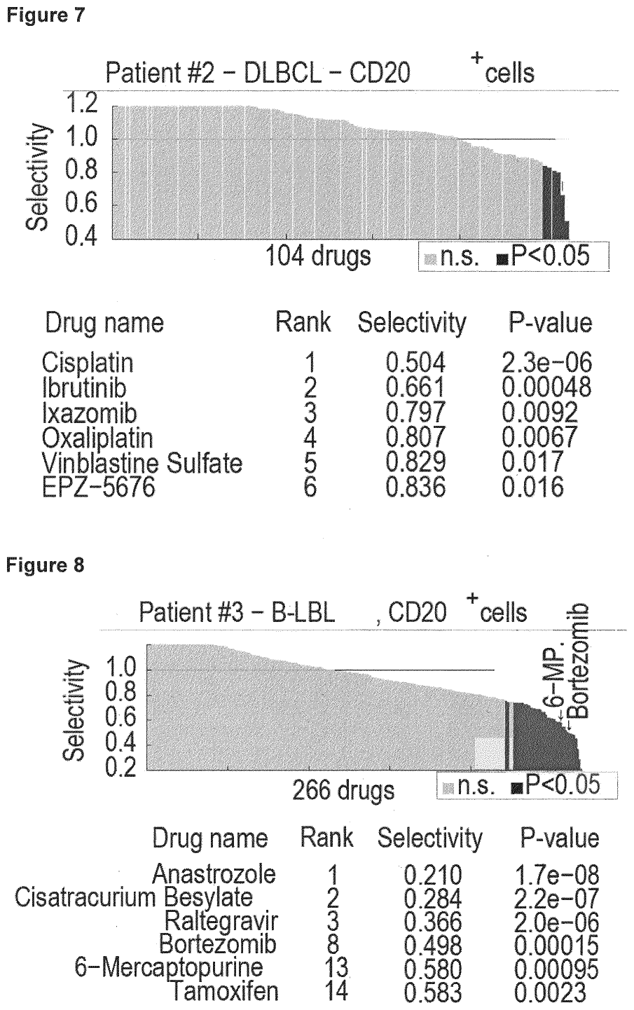

FIG. 7: Selectivities/values of FDA approved drugs to kill CD20+ cells over CD20− cells of a diffuse large B-cell lymphoma patient determined according to the present invention. The patient responded to treatment with ibrutinib.

FIG. 8: Selectivities/values of FDA approved drugs to kill CD20+ cells over CD20− cells of a B-cell lymphoblastic lymphoma patient determined according to the present invention. The patient responded to treatment with a combination of bortezomib and 6-mercaptopurine.

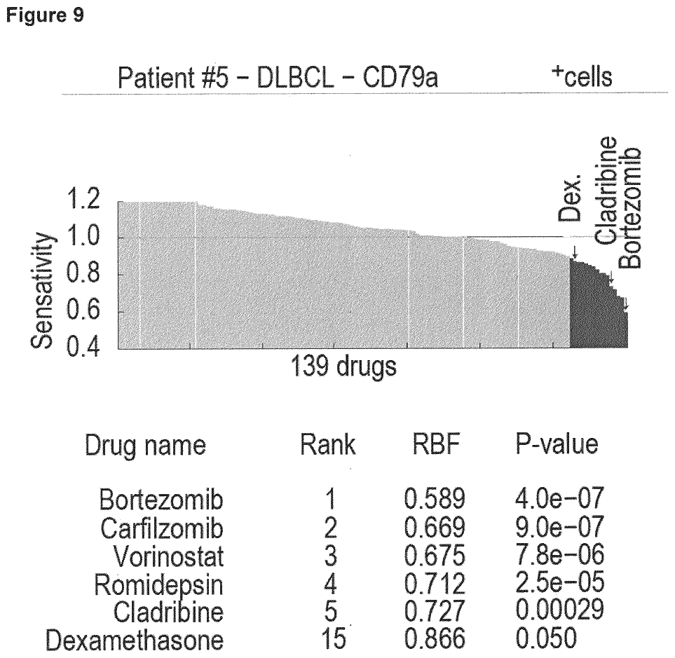

FIG. 9: Selectivities/values of FDA approved drugs to kill CD79a+ cells over CD79a− cells of a diffuse large B-cell lymphoma patient determined according to the present invention. The patient responded to treatment with a combination of bortezomib, cladribine and dexamethasone.

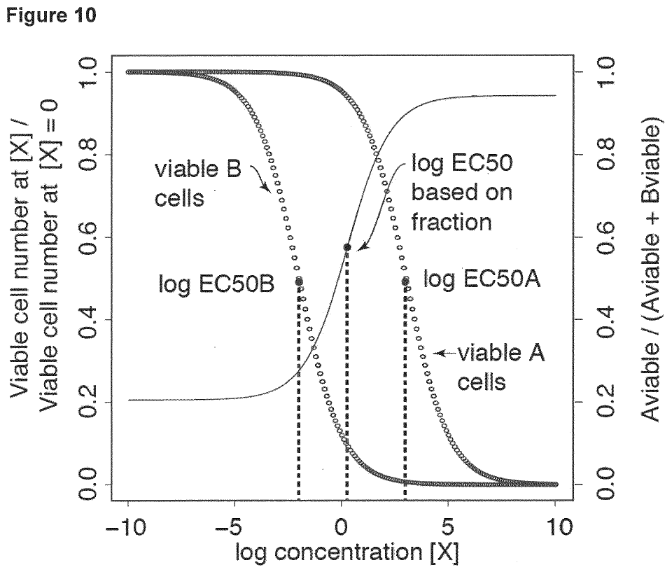

FIG. 10: The viability of cells of population A and population B after treatment with compound X at different concentrations [X] (log EC50 towards A=−2 and log EC50 towards B=3 on an arbitrar concentration scale) was calculated. Accordingly, the number of live cells of population A as a fraction of total live cells (population A+B) was determined as Aviable/(Aviable+Bviable). A logistic dose response curve was fit to the sigmoidal curve resulting from Aviable/(Aviable+Bviable) as a function of the concentration [X] (black line) and the log EC50 determined as the inflection point. Neither log EC50A nor log EC50B correspond to the log EC50 obtained from curve fitting (i.e., the log EC50 of the black line sigmoidal curve) demonstrating that a viability EC50A or EC50B cannot be obtained from fitting a logistic curve to Aviable/(Aviable+Bviable).

### EXAMPLES

### Example 1

Synthetic data simulating the response of a mixture of cells comprised of cell population A and B to a cytotoxic drug X was provided. X affected A with a log EC50 of EC50A (e.g., 2.5 for FIG. 1A) and B with a log EC50B (e.g., 3 for FIG. 1B) on an arbitrary concentration scale. Based on these parameters, the number of live cells and total number of cells of each type in the mixture of A and B cells could be calculated assuming a standard 4-parameter logistic (i.e., sigmoidal), dose-response curve. At a concentration of [X]=0, a total number of 10,000 cells at a ratio of A:B=0.8:0.2 was assumed. A measurement of the selectivity of X to kill A over B of a total number of only 3 drug concentrations was simulated.

Drug selectivity was calculated using the present invention. In particular, for each measured drug concentration the number of viable A cells and the number of viable B cells was calculated. According to step (d) of the methods of the invention (i) the number of viable cells in one of the at least two sub-populations (here A) that exhibit a distinguishable phenotype (here: viability), relative to the number of cells in the total population of cells (here viable A+viable B) that exhibit the same phenotype in the presence of X at three different concentrations as Rx=Ax/(Ax+Bx) where Ax and Bx denote the number of live A and B cells at three different concentrations [X] and (ii) for a concentration of [X]=0 giving R0=A0/(A0+B0) was determined. Then, the selectivity at each concentration of [X] as Sx=Rx/R0 was determined and averaged over all Sx to get the final selectivity of Sfinal=(S1+S2+S3)/3.

When determining the drug selectivity for different pairs of EC50A and EC50B using the present invention, surprisingly, it was linearly proportional to the difference in log EC50 of X towards A and the log EC50 of X towards B (FIG. 2).

### Example 2

Mononuclear cells were extracted from 20 bone marrow samples of treatment naïve patients newly diagnosed with acute myeloid leukemia (AML) using Ficoll density gradient centrifugation. After bone marrow samples had been taken, all 20 patients had undergone treatment with daunorucibin, etoposide and cytarabine according to the “3+5+7” schedule whereby 10 patients responded and 10 did not.

The mononuclear cells were suspended in RPMI+10% FCS+penicillin/streptomycin and were seeded into Perkin Elmer Cell Carrier 384-well cell culture plates at a concentration 20,000 cells in 50 μL medium per well whereby the wells had previously been loaded with combinations of cytarabine, daunorubicin and etoposide at different concentrations. All possible drug and concentration combinations were represented on the plate with cytarabine taking concentrations of 0, 1, 3, 10 and 20 μM, daunorubicin taking concentration of 0, 0.1, 1, 3 and 10 μM and etoposide taking concentrations of 0, 1, 3, 10 and 20 μM thus giving a 3-dimension drug titration matrix. Cells were allowed to form monolayers according to WO 2016/046346 and monolayers were incubated for 18 h, fixed by addition of 15 μL 4% formaldehyde solution in PBS containing 0.5% Tritox X114, flicked and stained with DAPI as well fluorescently labelled antibodies to mark CD34 and CD117 positive cells. After 1 h of incubation, images of each well were taken using an Opera Phenix automated confocal microscope (Perkin Elmer).

Marker positive cells were considered cancerous cells and marker negative cells as non-cancerous cells. The total number of live cells was quantified by counting intact DAPI-stained nuclei using the CellProfiler computational image analysis software whereas fragmented nuclei were discarded as dead or dying. Similarly, the number of live cancerous cells was determined as antibody stained cells with intact DAPI-stained nuclei.

The selectivity of each drug to kill the cancerous populations was determined according to the present invention by taking the fraction of live cancerous cells of live total cells relative to the fraction of live cancerous cells of live total cells in control wells (no drugs, just DMSO) for different concentrations of daunorubicin only (FIG. 3). Surprisingly, this measure alone could separate responders and non-responders.

To take into account the contribution of all drugs, the selectivity of each drug to kill the cancerous populations was determined according to the present invention by taking the fraction of live cancerous cells of live total cells at each drug combination and concentration relative to the fraction of live cancerous cells of live total cells in control wells (no drugs, just DMSO) and averaging over all concentrations and combinations of drugs. Using a cut-off value of 0.92, patients who clinically responded to the drug combination could be distinguished from patients who did not (FIG. 4, top panel) with a total classification accuracy of 0.85.

### Example 3

In analogy to Example 2, if drug response was determined only based on the sensitivity of cancer cells (here: CD34 or CD117 positive cells) or the sensitivity of the total cell population, a classification accuracy of 0.65 or less was obtained (FIG. 4, middle and bottom panels, respectively).

### Example 4

In analogy to Examples 2 and 3, this example illustrates how the selectivity determined according to the present invention can be used in downstream analysis to obtain a drug response score that allows for even more accurate classification of patients into responders and non-responders. For each patient sample and combination of drugs at different concentrations, the selectivity was calculated according to the present invention and averaged at each concentration point over responders and non-responders. The resulting data points span dose response surfaces in a four-dimensional dose-response space giving one surface for responders and one surface non-responders. The surface optimally separating the two dose response surfaces was determined by determining the cut-off point at each point in dose-response space that allowed for optimal classification into responders and non-responders at that particular drug dose combination. A response score was calculated for each patient by assigning a 1 to each point in dose response space, that was on the responders' side of the separating surface and a −1 that was on the opposite side. Summing over these indictors weighted by the total classification accuracy at each point in concentration space resulted in a final drug response score. This response score, for example, allowed AML patients receiving 3+5+7 induction therapy to be separated into responders and non-responders with over 90% total classification accuracy (FIG. 5) and area under the receiver operator curve (AUROC) of 0.97 whereas basing the same model on the number of cancerous cells normalized to the number of cancerous cells at zero drug concentration only, gave an AUROC of only 0.91 and basing it on cell number gave an AUROC of 0.86.

### Example 5

Bone marrow aspirates, peripheral blood, pleural effusion, ascites, or excised lymph node samples comprised of cells typically found in PBMCs or bone marrow were purified over Ficoll gradient (bone marrow, peripheral blood, pleural effusion, ascites) (GE healthcare) or homogenized and filtered (lymph tissue) and resuspended in RPMI+10% FCS and penicillin/streptomycin. The resulting single-cell suspensions of mononuclear cells commonly found in PBMCs were seeded in 384-well Perkin Elmer Cell Carrier imaging plates at a concentration of 20,000 cells in 50 μL medium per well plates according to WO 2016/046346 to form non-adherent monolayers. Plates had previously been loaded with 140 different clinically used anticancer drugs in 50 nL DMSO or 50 nL of DMSO as control such that each drug after addition of 50 μL medium and cells was present at 1 or 10 μM final concentration in at least 3 technical replicates per drug and concentration and the DMSO concentration amounted to 0.1% v/v.

Monolayers were incubated overnight (18 h). Biopsies used for the study were all freshly acquired and not stored frozen. Immunofluorescence staining, imaging by automated microscopy (Opera Phenix, Perkin Elmer), image analysis (CellProfiler), and data analysis (Matlab) were performed as described previously in Vladimer et al Nat Chem Biol 2017. The antibodies used to identify the target cancerous cell populations were selected based on clinical pathology reports and antibody reactivity assessment, and included CD3 (HIT3a), CD19 (HIB19), CD20 (2H7), CD79a (HM47), CD34 (4H11), CD117 (104ED2), and CD138 (DL-101) from eBiosciences. Unstained cells were considered non-cancerous cells.

The selectivity of drugs to kill cancer cells over non-cancerous cells was determined according to the present invention by taking the average fraction of live cancerous cells of live total cells for each drug and concentration relative to the average fraction of live cancerous cells of live total cells in control wells (no drugs, just DMSO). These quotients of average fractions over the two concentrations for each drug were determined.

Patients treated with drugs that displayed a value/selectivity of <1 as determined according to the present invention had a higher chance of responding (i.e., achieving a complete or partial remission) than patients treated with drugs that were chosen without taking the value as determined according to the present invention into account or drugs that had a value/selectivity of >1 as determined according to the present invention.

Moreover, when combinations of drugs were given to patients, the higher the sum of one minus the individual value (FIG. 6) of the drugs given to the patient, the higher the chance that the patient would respond.

### Example 6

A 69-year-old man with Diffuse Large B-cell lymphoma (DLBCL) relapsed after seven lines of prior treatment. Lymphoma cells of the sample were resistant to most of the 104 drugs tested as indicated by a selectivity of >1 of the drugs to kill the cancerous cells relative to non-cancerous cells as determined according the present invention, while only six compounds displayed significant on-target effects ex vivo (FIG. 7). Cisplatin and oxaliplatin were not considered feasible given the patient's history, age, and comorbidities, however the BTK inhibitor ibrutinib showed the second strongest ex vivo efficacy (value/selectivity according to the present invention=0.61, P<0.00048; FIG. 7). A PET-CT performed on day 49 of ibrutinib treatment confirmed a complete remission for the patient.

### Example 7

A 51-year-old women with precursor B-cell lymphoblastic lymphoma (B-LBL) had undergone three lines of prior treatment, and was progressive after immunotherapy with the bi-specific CD3-CD19 antibody blinatumomab. A cell mixture comprising cells typically found in PBMCs was isolated from, the woman's pleural effusion. The ability of 266 compounds to selectively kill cancerous versus non-cancerous cells contained in the cell mixture was determined using the present invention. It revealed that the proteasome inhibitor bortezomib was able to selectively kill cancer cancer cells (selectivity determined according to the present invention=0.50, P<0.001; FIG. 8) and the thiopurine 6-mercaptopurine, 6-MP (value/selectivity determined according to the present invention=0.58, P<0.001; FIG. 8). 6-MP and bortezomib were combined with anti-CD20 obinutuzumab. After 28 days PET-CT confirmed a partial response.

### Example 8

An excised lymph node of a patient with diffuse large B-cell lymphoma was dissociated into single cells giving a complex cell mixture comprising cells typically found in PBMCs. The ability of 266 compounds to selectively kill cancerous versus non-cancerous cells contained in the cell mixture according to the present invention. The patient achieved a complete remission (FIG. 9) to a combination of the single strongest ex vivo acting drug, bortezomib (selectivity=0.59, P<0.0001;), with cladribine (selectivity=0.73; P<0.0003) and dexamethasone (value/selectivity=0.87; P<0.05; FIG. 9).

### Example 9

This example describes the practical application of the methods described in claims (1) and following and in particular of claims 1) and (2). A tissue sample of a blood cancer patient comprised of 40,000 cells is provided. 20,000 cells are cancerous cells and stain positive for the cell surface marker CD19. The remaining cells stain positive for other cell surface markers including CD3, 4, 8, 11c, 14, 56 and others. The sample is divided into two parts of 20,000 cells each. The first sample is incubated in RPMI+10% FCS in the presence of 10 μM bortezomib in DMSO (0.1% final DMSO concentration) whereas the second sample is incubated in RPMI+10% FCS+0.1% DMSO. After 24 h incubation the viability of each cell in each sample is determined whereby viability here is the “distinguishable phenotype” referred to in step (d) of claim 1 and dependent claims. In the bortezomib treated sample, 5,000 viable cells staining for CD19 are found and 10,000 viable cells negative for CD19 stain are found. In the DMSO treated sample 10,000 viable cells staining positive for CD19 and staining negative for CD19 are found each. According to the present invention, the selectivity of bortezomib to reduce viability of the CD19 positive cells is calculated. Following step (d) the number of cells in one of the at least two sub-populations (here: CD19 positive cells) that exhibit a distinguishable phenotype (here: viability), relative to the number of cells in the total population of cells (CD19 positive+CD19 negative cells) that exhibit the same phenotype (i.e., viability) in (i) the at least one part incubated in the presence of bortezomib as the test compound (here: 5,000/15,000=0.33) and (ii) in the at least one part incubated in the absence of the test compound (here: 10,000/20,000=0.5) is calculated.

Following step (e) the selectivity of the test compound (here: bortezomib) to induce the phenotype referred to in (d) in the one sub population referred to in step (d) (here: CD19 positive cells) over all other subpopulations is determined by dividing (i) (here: 0.33) through (ii) (here: 0.50). Since 0.33/0.50=0.66 i.e., less than 1, the test compound (here: bortezomib) selectively inhibits the phenotype of steps (d) (here: viability) in the one population explicitly referred to in step (d) (here: CD19 positive cells). So, according to the present invention we can conclude that bortezomib selectively reduced the viability of CD19 positive cells in the given example.

### Example 10

This example describes a further practical application of the methods of the invention. A tissue sample of a blood cancer patient comprised of 60,000 cells is provided. 30,000 cells are cancerous cells and stain positive for the cell surface marker CD79a. The remaining cells stain positive for other cell surface markers including CD3, 4, 8, 11c, 14, 56 and others. Suitably labelled antibodies are used as staining reagents.

The sample is divided into two parts of 40,000 cells and 20,000 cells. The first part of 40,000 cells is further divided into two parts of 20,000 cells each here denoted [1a] and [1b]. Parts [1a] and [1b] are incubated in RPMI+10% FCS in the presence of 10 μM and 1 μM bortezomib in DMSO (0.1% final DMSO concentration) respectively whereas the second sample is incubated in RPMI+10% FCS+0.1% DMSO. CD79a here is only chosen as a hypothetical example for the sake of clarity and can be replaced with any other surface marker.

After 24 h incubation the viability of each cell in each sample is determined whereby viability here is the “distinguishable phenotype” as used in the present invention. In the bortezomib treated sample [1a], 5,000 viable cells staining for CD79a are found and 10,000 viable cells negative for CD79a stain are found. In the bortezomib treated sample [1b], 8,000 viable cells staining for CD79a are found and 10,000 viable cells negative for CD79a stain are found. In the DMSO treated sample 10,000 viable cells staining positive for CD79a and staining negative for CD79a are found each. The selectivity of bortezomib to reduce viability of the CD79a positive cells is calculated according to the methods of the invention.

For both parts [1a] and [1b] the number of, cells in the one of the at least two sub-populations (here: CD79a positive cells) that exhibit a distinguishable phenotype (here: viability), relative to the number of cells in the total population of cells (CD79a positive+CD79a negative cells) that exhibit the same phenotype (i.e., viability) in (i) the at least one part incubated in the presence of bortezomib at the respective concentration as the test compound (here: 5,000/15,000=0.33 for [1a] and 8,000/18,000=0.44 for [1b]) and (ii) in the at least one part incubated in the absence of the test compound (here: 10,000/20,000=0.5) is calculated.

Following step (e) the selectivity of the test compound (here: bortezomib) to induce the phenotype referred to in (d) in the one sub-population referred to in step (d) (here: CD79a positive cells) over all other subpopulations is determined by dividing (i) (here: 0.33 for [1a] and 0.44 for [1b]) through (ii) (here: 0.50) and the average selectivity is calculated as the final value/selectivity as (0.33/0.50+0.44/0.50)/2=0.77. Since 0.77 is less than 1, the test compound (here: bortezomib) selectively inhibits the phenotype of steps (d) (here: viability) in the one population explicitly referred to in step (d) (here: CD79 positive cells). So, according to the present invention it can be concluded that bortezomib selectively reduced the viability of CD79a positive cells in the given example.

The patient from wham the sample was derived would respond to treatment with bortezomib. CD79a here is only chosen as a hypothetical example for the sake of clarity and can be replaced with any other surface marker. Also cell numbers are only chosen arbitrarily for illustrative purposes.

### Example 11

A tissue sample of a blood cancer patient comprised of 60,000 cells is provided. 30,000 cells are cancerous cells and stain positive for the cell surface marker CD20. The remaining cells stain positive for other cell surface markers including CD3, 4, 8, 11c, 14, 56 and others. Suitably labelled antibodies are used as staining reagents. The sample is divided into three parts of 20,000 cells each. Two parts of 20,000 each are incubated in RPMI+10% FCS in the presence of 10 μM bortezomib in DMSO (0.1% final DMSO concentration) respectively whereas the third part is incubated in RPMI+10% FCS+0.1% DMSO. Please note that CD20a here is only chosen as a hypothetical example for the sake of clarity and can be replaced with any other surface marker.

After 24 h incubation the viability of each cell in each sample is determined whereby viability here is the “distinguishable phenotype”. In the two samples treated with 10 μM bortezomib, 5,000 viable cells staining for CD20a are found and 10,000 viable cells negative for CD20a stain are found each. In the DMSO treated sample 10,000 viable cells staining positive for CD20 and staining negative for CD20 are found each. The selectivity of bortezomib to reduce viability of the CD79a positive cells is determined. For both parts incubated in the presence of 10 μM bortezomib the number of cells in the one of the at least two sub-populations, (here: CD20 positive cells) that exhibit a distinguishable phenotype (here: viability), relative to the number of cells in the total population of cells (CD20 positive+CD20 negative cells) that exhibit the same phenotype (i.e., viability) in (i) each part incubated in the presence of bortezomib is calculated independently (i.e., 5,000/15,000=0.33 and 5,000/15,000=0.33) and (ii) for each part incubated in the absence of the test compound (here: 10,000/20,000=0.5) is determined independently. Then the average of (i) and (ii) are formed giving 0.33 for (i) and 0.5 for (ii) and used for further steps, that is, in step (e) the value/selectivity is determined by dividing the average of (i) by the average of (ii) giving 0.33/0.5=0.66 as the final value/selectivity.

### Example 12

This example illustrates that accurate EC50 values cannot be obtained from fitting dose response curves to fractions of cells exhibiting a phenotype of total cells exhibiting the same phenotype. A mixture of cells of type A and B at a ratio of A:B=0.2:0.8 was assumed. The cell mixture was treated with a cytotoxic compound X. The ability of compound X to kill A cells was quantified with a log EC50 of 3 and the ability of compound X to kill B cells was quantified with a log EC50 of −2 on an arbitrary concentration scale. Calculating the fraction of the number live A cells of the total number of live cells (i.e. live A+live B), the sigmoidal curve shown in FIG. 10 was obtained. It can be clearly seen, that the inflection point of this curve (solid line) is neither informative of the EC50 of the dose response curve of X action on A nor of X action on B.

### Example 13

This example illustrates the effect of a 10% standard deviation in total cell numbers when introducing a cell mixture of A and B cells into a microtiter plate for determining selectivity of a test compound X to kill A over B cells. When determining the selectivity using the classic approach of measuring total number of A and B cells as a function of concentration [X], to fit sigmoidal dose response curves and measure the EC50 of X towards A and B, each measurement point will have a standard deviation of 10%. Using the present invention, a 10% variation in total cell number will have no effect on the fraction of viable A cells of the total number of viable cells. The present invention thus allows for the determination of selectivity that is more robust towards variation in seeding of cells into assay plates or loss of cells during manipulation.

1. A method for determining the selectivity of a test compound, the method comprising the steps:
(a) providing a sample comprising at least two distinguishable sub-populations of cells in a total population of cells;
(b) dividing the sample into at least two parts;
(c) incubating at least one part obtained in step (b) in the absence of a test compound and at least one part obtained in step (b) in the presence of a test compound;
(d) determining the number of cells in one of the at least two sub-populations that exhibit a distinguishable phenotype, relative to the number of cells in the total population of cells that exhibit the same phenotype in
(i) the at least one part incubated in the presence of the test compound and
(ii) in the at least one part incubated in the absence of the test compound; and
(e) determining selectivity of the test compound to induce the phenotype referred to in (d) in the one sub population referred to in step (d) over all other subpopulations by dividing (i) through (ii) wherein the test compound selectively induces the phenotype referred to in (d) if (i) divided through (ii) is greater than 1, preferably greater than 1.05, 1.1, 1.5, 2, 3 most preferably 5, and selectively inhibits or reduces the phenotype referred to in (d) if it is less than 1, preferably less than 0.95, 0.9, 0.7, 0.5, 0.3, most preferably less than 0.2.

2. The method according to claim 1, wherein the distinguishable phenotype in step (d) is viability and wherein
(i) if the selectivity determined in step (e) is <1 the test compound is determined to selectively reduce the number of viable cells of the one sub-population of step (d), and
(ii) if the selectivity determined in step (e) is >1 the test compound is determined to selectively improve viability of the one sub-population and/or to selectively reduce the viability of one or more of the sub-population(s) other than the one sub-population of step (d).

3. A method for determining whether a subject suffering from cancer will respond or is responsive to treatment with a test compound, the method comprising the steps:
(a) providing a sample obtained from the subject comprising at least two sub-populations of cells in a total population cells, wherein at least one sub-population corresponds to cancerous cells and at least one sub-population corresponds to non-cancerous cells;
(b) dividing the sample into at least two parts;
(c) incubating at least one part obtained in step (b) in the absence of a test compound and at least one part in the presence of a test compound;
(d) determining the number of viable cells in at least one of the sub-populations corresponding to cancer cells relative to the number of viable cells in the total population of cells in
(i) the at least one part incubated in the presence of the test compound and
(ii) the at least one part incubated in the absence of the test compound; and
(e) determining whether the subject will respond or is responsive to treatment with the test compound by dividing (i) through (ii), wherein the subject will respond or is responsive to treatment if the resulting value is less than 1, preferably less than 0.95, 0.9, 0.8, 0.6, 0.4 most preferably less than 0.2.

4. The method of claim 3, wherein the method is repeated for at least two test compounds and whether the subject will respond or is responsive to treatment with a combination of the at least two test compounds is determined by subtracting the values obtained in (e) for each of the at least two test compounds from 1.0, and summing over the resulting values for the at least two test compounds wherein if the resulting sum is greater than −1, preferably greater than −0.5, 0, 0.5, most preferably greater than 1, the subject is determined to respond or be responsive to treatment with the combination of the at least two test compounds.

5. The method according to claim 1, wherein the test compound(s) comprise(s) one or more chemical substances.

6. The method according to claim 1, wherein at least one part obtained in step (b) is further divided into at least two parts, wherein each of the at least two parts is incubated in step (c) with the test compound at different concentrations and wherein steps (d) and (e) are repeated for each concentration of the test compound independently to determine a selectivity/value at each concentration of the test compound whereby an average selectivity/value over all concentrations is calculated after step (e) and used for determining the final selectivity/value.

7. The method according to claim 1, wherein in step (b) the sample is divided into at least three parts and in step (c) at least two parts are incubated in the absence of a test compound and/or at least two parts are incubated in the presence of a test compound whereby each part incubated in the presence of the test compound is incubated in the presence of the same concentration of the test compound, and wherein in step (d) the number of cells in the one of the at least two sub-populations that exhibit a distinguishable phenotype relative to the number of cells in the total population of cells that exhibit the same distinguishable phenotype is determined for
(i) each part incubated in the presence of the test compound independently and/or
(ii) each part incubated in the absence of the test compound independently and the average of the relative numbers obtained in (i) and/or the average of the relative numbers obtained in (ii) is used.

8. The method according to claim 1, wherein in step (b) the sample is divided into at least three parts and in step (c) at least one part is incubated in the absence of a test compound and/or at least two parts are incubated in the presence of at least two different concentrations of the test compound and in step (d) the number of cells in the one of the at least two sub-populations that exhibit a distinguishable phenotype relative to the number of cells in the total population of cells that exhibit the same distinguishable phenotype is determined for
(i) each part incubated in the presence of the test compound independently and/or
(ii) each part incubated in the absence of the test compound independently
wherein the average of (i) is determined for each concentration independently and/or the average of (ii) is determined and used for further steps and wherein in step (e) the selectivity/value is determined for each concentration of the test compound by dividing the average of (i) for each concentration through the average of (ii) and the final selectivity/value is obtained by averaging the selectivity/value for each concentration.

9. The method according to claim 3, wherein the method is repeated for at least two test compounds and the test compound with the lowest value obtained in step (e) is selected for treatment of the subject suffering from cancer.

10. The method according to claim 4, wherein the method is repeated for at least three test compounds and the combination of at least two of the at least three test compounds with the highest value obtained by subtracting the values obtained in (e) for each of the at least two test compounds in the combination from 1.0, and summing over the resulting values for the at least two test compounds in the combination, is selected for treatment of the subject suffering from cancer.

11. The method according to claim 3, wherein the cancer is a cancer associated with PBMCs or bone marrow cells or cells derived from PBMCs or bone marrow cells.

12. The method according to claim 1, wherein the sample is a tissue sample that contains at least 1% cancerous cells and/or at least 1% non-cancerous cells.

13. The method according to claim 12, wherein the tissue sample is cultured as a non-adherent cell monolayer.

14. The method according to claim 13, wherein the number of viable cancerous and non-cancerous cells is determined using automated microscopy.

15. The method according to claim 14, wherein the number of viable cells is determined as the number of non-fragmented nuclei.
